(19) United States
(12) Patent Application Publication
(10) Pub. No.: US 2022/0096896 A1
(43) Pub. Date: Mar. 31, 2022

(54) MEASUREMENT DEVICE, MEASUREMENT METHOD, AND RECORDING MEDIUM

(71) Applicant: CASIO COMPUTER CO., LTD., Tokyo (JP)

(72) Inventor: Hiroki TOMITA, Tokyo (JP)

(73) Assignee: CASIO COMPUTER CO., LTD., Tokyo (JP)

(21) Appl. No.: 17/465,912

(22) Filed: Sep. 3, 2021

(30) Foreign Application Priority Data
Sep. 28, 2020 (JP) 2020-161650

(51) Int. Cl. A63B 24/00

(52) U.S. Cl. CPC A63B 24/0003; A63B 2024/0068; A63B 2024/0012; A63B 24/0062

(57) ABSTRACT

A measurement device includes at least one processor that acquires acceleration data indicating a temporal transition of acceleration of a subject when the subject is moving by performing a moving action, detects a target timing that is a timing when the subject performs a target action in accordance with a temporal transition of the acceleration of the subject in a detection target direction, the temporal transition being indicated by the acceleration data, and in accordance with reference data indicating a temporal transition of the acceleration of the subject when the subject performs the target action in the moving action, detects the target timing using a first detection method when the moving action is a first moving action, and detects the target timing using a second detection method different from the first detection method when the moving action is a second moving action different from the first moving action. At least one of the detection target direction or the reference data is different between when the target timing is detected using the first detection method and when the target timing is detected using the second detection method.

### BACKGROUND

### 1. Technical Field

The present invention relates to a measurement device, a measurement method, and a recording medium.

### 2. Related Art

There is known a measurement device that detects a timing at which a running user performs a specific action, and acquires information regarding a manner of running of the user in accordance with the detected timing. For example, JP 2017-169837 A discloses a measurement device that detects a landing timing at which a foot of a running user touches the ground and a takeoff timing at which the foot of the running user leaves the ground, and calculates a contact time in accordance with the detected landing timing and takeoff timing.

### SUMMARY

A measurement device of the present invention includes at least one processor configured to execute a program stored in at least one memory, in which

the at least one processor acquires acceleration data indicating a temporal transition of acceleration of a subject when the subject is moving by performing a moving action,

the at least one processor detects a target timing that is a timing when the subject performs a target action in accordance with a temporal transition of the acceleration of the subject in a detection target direction, the temporal transition being indicated by the acceleration data, and in accordance with reference data indicating a temporal transition of the acceleration of the subject when the subject performs the target action in the moving action,

the at least one processor detects the target timing using a first detection method when the moving action is a first moving action,

the at least one processor detects the target timing using a second detection method different from the first detection method when the moving action is a second moving action different from the first moving action, and

at least one of the detection target direction or the reference data is different between when the target timing is detected using the first detection method and when the target timing is detected using the second detection method.

### BRIEF DESCRIPTION OF THE DRAWINGS

FIG. 1 is a diagram illustrating an overall configuration of a measurement system according to an embodiment of the present invention;

FIG. 2 is a diagram illustrating a physical configuration of a detection device according to the embodiment of the present invention;

FIG. 3 is a diagram illustrating a functional configuration of the detection device according to the embodiment of the present invention;

FIG. 4 is a diagram illustrating an example of a waveform representing a temporal transition of a position of a waist of a user according to the embodiment of the present invention;

FIG. 5 is a diagram illustrating an example of a cumulative cost map according to the embodiment of the present invention;

FIG. 6 is a diagram illustrating a physical configuration of a terminal device according to the embodiment of the present invention;

FIG. 7 is a diagram illustrating a functional configuration of the terminal device according to the embodiment of the present invention;

FIG. 8 is a diagram illustrating a physical configuration of an information processor according to the embodiment of the present invention;

FIG. 9 is a flowchart for describing estimator creation processing executed by the information processor according to the embodiment of the present invention;

FIG. 10 is a flowchart for describing prefilter creation processing executed by the information processor according to the embodiment of the present invention;

FIG. 11 is a flowchart for describing template creation processing executed by the information processor according to the embodiment of the present invention;

FIG. 12 is a flowchart for describing control processing executed by the detection device according to the embodiment of the present invention;

FIG. 13 is a flowchart for describing output processing executed by the detection device according to the embodiment of the present invention;

FIG. 14 is a flowchart for describing timing detection processing executed by the detection device according to the embodiment of the present invention;

FIG. 15 is a flowchart for describing dynamic time warping processing executed by the detection device according to the embodiment of the present invention; and

FIG. 16 is a flowchart for describing presentation processing executed by the terminal device according to the embodiment of the present invention.

### DETAILED DESCRIPTION

Hereinafter, an embodiment of the present invention will be described with reference to the drawings. In the drawings, the same or equivalent components are denoted by the same reference numerals.

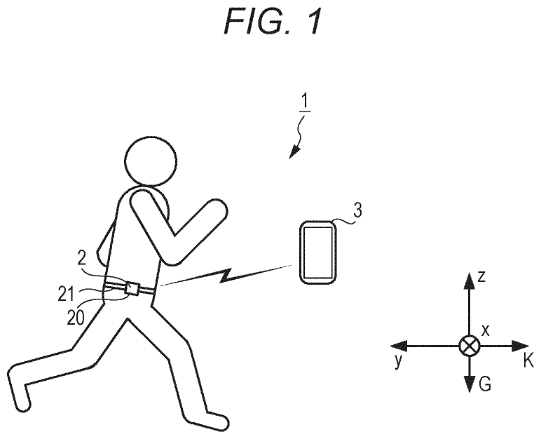

When a user of a detection device 2 who owns the detection device 2 is running, that is, when the user is moving by performing a running action, a measurement system 1 illustrated in FIG. 1 acquires running action information that indicates a feature of the running action performed by the user, and presents the acquired running action information to the user. The user of the detection device 2 is an example of a subject. The running action is an example of a moving action. The running action information is an example of moving action information. Note that, in this embodiment, the subject is described as the user of the detection device 2 who owns the detection device 2, but this is merely an example. The subject may be a human who borrows the detection device 2 from a user of the detection device 2 who owns the detection device 2, and uses the detection device 2.

As illustrated in FIG. 1, the measurement system 1 includes a detection device 2 attached to the waist of the user and a terminal device 3 carried by the user. The detection device 2 is an example of a measurement device. The detection device 2 includes a body 20 and a belt 21 wound around the waist of the user. The body 20 of the detection device 2 is fixed to a center of the waist of the user by the belt 21. When the user is running, the detection device 2 detects acceleration of the running user, that is, a temporal transition of acceleration of the user's body. The detection device 2 detects the takeoff timing at which the user performs a takeoff action when the user is running in accordance with the detected temporal transition of the acceleration of the user. The takeoff action is an action of taking at least one of the feet off from the ground. When performing the running action, the user alternately and repeatedly executes the takeoff action and a landing action to be described later. That is, the takeoff action and the landing action are actions included in the running action and related to the running action. The takeoff action is an example of a target action. The takeoff timing is an example of a target timing. The detection device 2 acquires the running action information according to the detected takeoff timing, and transmits the acquired running action information to the terminal device 3.

In the description of this embodiment, the user moves by performing the running action, but this is merely an example. The user can move by performing any moving action. For example, the user may move by performing a walking action. In this case, the detection device 2 may detect the temporal transition of the acceleration of the walking user, detect the takeoff timing at which the walking user performs the takeoff action in accordance with the temporal transition of the detected acceleration of the user, and acquire walking action information that indicates the feature of the walking action of the user in accordance with the detected takeoff timing. The walking action is an example of the moving action, and the walking action information is an example of the moving action information. Further, in the description of this embodiment, the detection device 2 is fixed to the waist of the user, but this is merely an example, and the detection device 2 can be fixed to any part of the body of the user. For example, the detection device 2 may be fixed to the chest of the user. Alternatively, the detection device 2 may be fixed to the abdomen of the user. Further, in the description of this embodiment, the detection device 2 is fixed to the waist of the user by the belt 21, but this is merely an example, and the detection device 2 can be fixed to the waist of the user by any method. For example, the detection device 2 may be fixed to the waist of the user by a clip that sandwiches the user's clothes.

The terminal device 3 is a smartphone, receives the running action information from the detection device 2, and presents the received running action information to the user. In this embodiment, the terminal device 3 is described as a smartphone, but this is merely an example, and the terminal device 3 may be any information processing terminal device such as a tablet terminal, a smart watch, or a personal computer (PC). In the description of this embodiment, the detection device 2 acquires and transmits the running action information to the terminal device 3, and the terminal device 3 receives and presents the running action information to the user. However, this is merely an example, and the detection device 2 may present the acquired running action information to the user.

In the description of this embodiment, the detection device 2 functions as the measurement device of the present invention, but this is merely an example, and the terminal device 3 may function as the measurement device of the present invention. In this case, the terminal device 3 only has to acquire data indicating the temporal transition of the acceleration of the user detected by the detection device 2 from the detection device 2, execute processing such as detection of the takeoff timing and acquisition of the running action information executed by the detection device 2 in this embodiment on the basis of the acquired data, and present the acquired running action information to the user. Alternatively, the measurement system 1 may include an information processor such as a PC or a server different from both the detection device 2 and the terminal device 3, and the information processor may function as the measurement device of the present invention. In this case, the information processor only has to acquire data indicating the temporal transition of the acceleration of the user detected by the detection device 2 from the detection device 2, and execute processing such as detection of the takeoff timing and acquisition of the running action information executed by the detection device 2 in this embodiment on the basis of the acquired data. The information processor only has to transmit the acquired running action information to the terminal device 3, and the terminal device 3 only has to present the running action information received from the information processor to the user.

Hereinafter, xyz coordinate axes illustrated in FIG. 1 are set in order to facilitate understanding. In FIG. 1, an arrow G indicates a vertical direction, that is, a direction of gravitational acceleration. Further, in FIG. 1, an arrow K indicates an advancing direction of the user, that is, a direction from the back of the user's body toward the front of the user's body. An x-axis direction, a y-axis direction, and a z-axis direction illustrated in FIG. 1 are orthogonal to each other. The x-axis direction is a direction orthogonal to the advancing direction of the user. An x-axis positive direction is a direction from the right to the left in the advancing direction of the user, and an x-axis negative direction is a direction from the left to the right in the advancing direction of the user. The y-axis direction is a direction parallel to the advancing direction of the user. A y-axis positive direction is the advancing direction of the user, and a y-axis negative direction is a direction opposite to the advancing direction of the user. The z-axis direction is a direction parallel to the vertical direction. A z-axis positive direction is a direction opposite to the vertical direction, and a z-axis negative direction is the vertical direction.

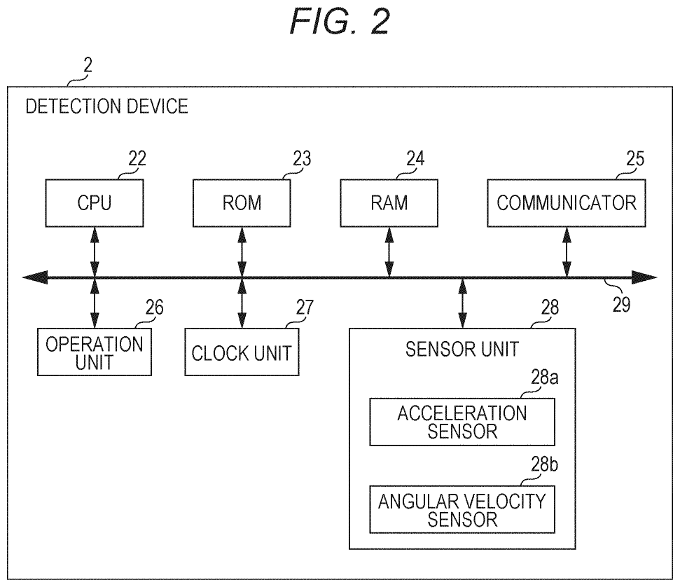

Hereinafter, physical and functional configurations of the detection device 2 will be described with reference to FIGS. 2 to 5. First, a physical configuration of the detection device 2 will be described with reference to FIG. 2. As illustrated in FIG. 2, the detection device 2 includes a central processing unit (CPU) 22 as at least one processor, a read only memory (ROM) 23 as at least one memory, a random access memory (RAM) 24, a communicator 25, an operation unit 26, a clock unit 27, and a sensor unit 28 in addition to the body 20 and the belt 21 described above. The CPU 22 to the sensor unit 28 are interconnected via a system bus 29 as a command and data transmission path. The CPU 22 to the sensor unit 28 are built in the body 20. Note that FIG. 2 illustrates only a configuration related to a characteristic part of the present invention in the physical configuration included in the detection device 2. The detection device 2 may include any physical configuration not illustrated in FIG. 2. For example, the detection device 2 may include a display device that displays various images.

The CPU 22 controls each unit of the detection device 2 in accordance with a program and data stored in the ROM 23 and executes various processing. The ROM 23 stores, in a non-transitory manner, a program and data used by the CPU 22 to execute various processing. The RAM 24 functions as a work area of the CPU 22. That is, the CPU 22 reads the program and data stored in the ROM 23 to the RAM 24, and executes various processing by referring to the read program and data. In addition, the CPU 22 temporarily stores data acquired by executing various processing in the RAM 24, and executes various processing by referring to the stored data. The communicator 25 performs wireless communication or wired communication with an external device including the terminal device 3 under the control of the CPU 22, and transmits and receives data. The communicator 25 outputs the data received from the external device to the CPU 22. The operation unit 26 includes a plurality of keys that receives an operation by the user, including a detection device power key that receives an operation of switching on and off a power source of the detection device 2, detects the operation on the plurality of keys by the user, and outputs an operation signal indicating a detection result to the CPU 22. The clock unit 27 includes a real time clock (RTC), clocks a current time, and outputs a clock signal indicating a clock result to the CPU 22. The sensor unit 28 detects acceleration and angular velocity of the user's body. The sensor unit 28 includes an acceleration sensor 28a and an angular velocity sensor 28b. The acceleration sensor 28a detects accelerations in three axial directions orthogonal to each other, and the angular velocity sensor 28b detects angular velocities around the three axial directions. The acceleration sensor 28a sequentially transmits an acceleration signal that indicates a detection result of the acceleration to the CPU 22. The angular velocity sensor 28b sequentially transmits an angular velocity signal that indicates a detection result of the angular velocity to CPU 22.

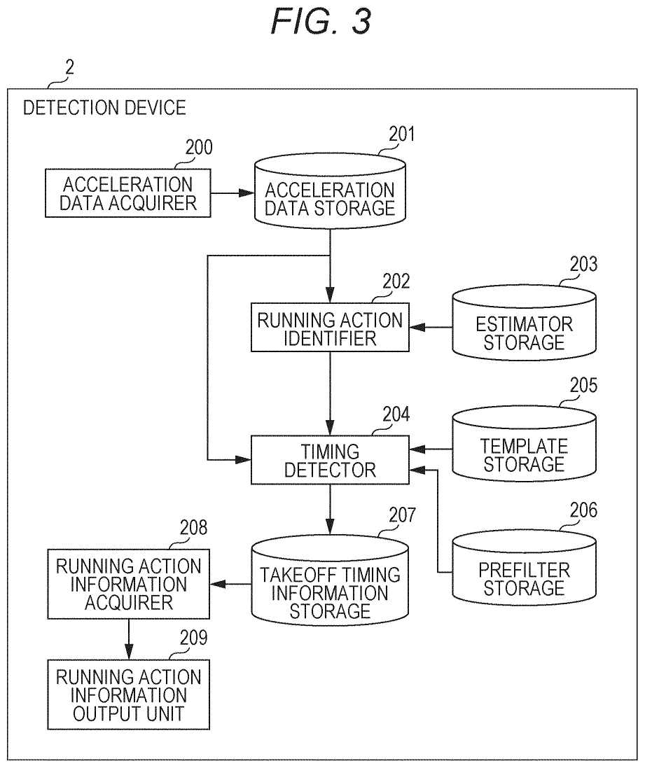

As illustrated in FIG. 3, the detection device 2 having the physical configuration described above functionally includes an acceleration data acquirer 200, an acceleration data storage 201, a running action identifier 202, an estimator storage 203, a timing detector 204, a template storage 205, a prefilter storage 206, a takeoff timing information storage 207, a running action information acquirer 208, and a running action information output unit 209. The acceleration data acquirer 200, the running action identifier 202, the timing detector 204, the running action information acquirer 208, and the running action information output unit 209 are realized by the CPU 22. That is, the CPU 22 executes the program stored in the ROM 23 to control each unit of the detection device 2, and thus functions as the acceleration data acquirer 200, the running action identifier 202, the timing detector 204, the running action information acquirer 208, and the running action information output unit 209. The estimator storage 203, the template storage 205, and the prefilter storage 206 are realized by the ROM 23. That is, the estimator storage 203, the template storage 205, and the prefilter storage 206 are constructed in a storage area of the ROM 23. The acceleration data storage 201 and the takeoff timing information storage 207 are realized by the RAM 24. That is, the acceleration data storage 201 and the takeoff timing information storage 207 are constructed in a storage area of the RAM 24. Note that FIG. 3 illustrates only a functional configuration related to a characteristic part of the present invention in the functional configuration included in the detection device 2. The detection device 2 may include any functional configuration not illustrated in FIG. 3. For example, the detection device 2 may include a display controller that controls display of an image by the display device described above.

The acceleration data acquirer 200 acquires acceleration data indicating the temporal transition of the acceleration of the user in accordance with the acceleration signal input from the acceleration sensor 28a. Specifically, the acceleration data acquirer 200 uses Kalman filter to estimate the vertical direction and the advancing direction of the user in accordance with the detection result of the acceleration indicated by the acceleration signal input from the acceleration sensor 28a and the detection result of the angular velocity indicated by the angular velocity signal input from the angular velocity sensor 28b, and thus converts an acceleration value in a sensor coordinate system detected by the acceleration sensor 28a into an acceleration value in a world coordinate system defined by the above xyz coordinate axes. The acceleration data acquirer 200 acquires acceleration data by sampling the converted acceleration value at a predetermined sampling frequency (in this embodiment, 200 Hz). The acceleration data acquired in this manner includes data of a plurality of sampling points in chronological order, and data of one sampling point included in the acceleration data indicates the acceleration of the user detected by the acceleration sensor 28a at the one sampling point. The acceleration data acquired by the acceleration data acquirer 200 includes x-axis acceleration data that indicates a temporal transition of the acceleration of the user in the x-axis direction, y-axis acceleration data that indicates a temporal transition of the acceleration of the user in the y-axis direction, and z-axis acceleration data that indicates a temporal transition of the acceleration of the user in the z-axis direction. That is, the acceleration data including the x-axis acceleration data, the y-axis acceleration data, and the z-axis acceleration data indicates the temporal transition of the acceleration of the user in the x-axis direction, the y-axis direction, and the z-axis direction. The y-axis acceleration data is an example of first acceleration data, and the z-axis acceleration data is an example of second acceleration data.

Note that, in the description of this embodiment, the acceleration data acquirer 200 converts the acceleration value in the sensor coordinate system detected by the acceleration sensor 28a into the acceleration value in the world coordinate system defined by the xyz coordinate axes with use of the Kalman filter, but this is merely an example. The acceleration data acquirer 200 can convert the acceleration value in the sensor coordinate system detected by the acceleration sensor 28a into the acceleration value in the world coordinate system by any method. For example, the sensor unit 28 may include a geomagnetic sensor that detects geomagnetism in three axial directions orthogonal to each other in addition to the acceleration sensor 28a and the angular velocity sensor 28b, and the acceleration data acquirer 200 may convert the acceleration value in the sensor coordinate system detected by the acceleration sensor 28a into the acceleration value in the world coordinate system by estimating the vertical direction in accordance with a detection result of the geomagnetism by the geomagnetic sensor.

The acceleration data storage 201 stores the acceleration data acquired by the acceleration data acquirer 200. The acceleration data acquirer 200 acquires acceleration data corresponding to an acceleration signal every time the acceleration signal is input from the acceleration sensor 28a, and stores the acquired acceleration data in the acceleration data storage 201. With such a configuration, the acceleration data acquired by the acceleration data acquirer 200 is accumulated in the acceleration data storage 201.

In accordance with the acceleration data acquired by the acceleration data acquirer 200, the running action identifier 202 identifies whether the running action performed by the user is a first running action or a second running action of which the takeoff timing is later than the takeoff timing of the first running action. The first running action is a running action in a case where the user is running at a first reference speed (in this embodiment, 2.5 m/s) as an average running speed of adults. The second running action is a running action in a case where the user is running at a low speed, that is, at a second reference speed (in this embodiment, 2.0 m/s) lower than the first reference speed described above. When the user is moving by performing the first running action, both feet of the user do not simultaneously contact the ground. On the other hand, when the user is moving by performing the second running action, both feet of the user may simultaneously contact the ground. The first running action is an example of a first moving action. The second running action is an example of a second moving action.

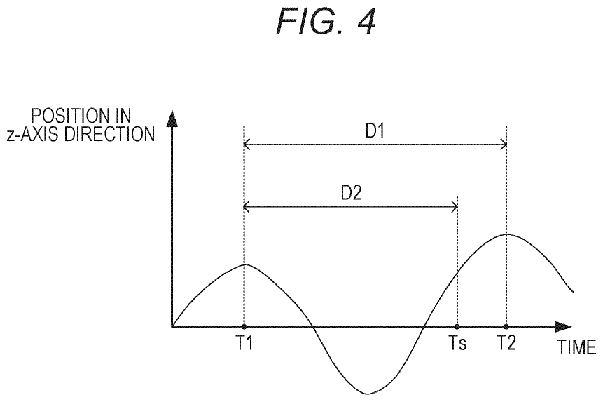

Hereinafter, a difference between the first running action and the second running action will be described with reference to FIG. 4. FIG. 4 illustrates an example of a waveform representing a temporal transition of a position of the waist of the user in the z-axis direction. In FIG. 4, T1 indicates a first maximum value timing at which the position of the waist of the user in the z-axis direction becomes a maximum value in the temporal transition of the position of the waist of the user in the z-axis direction. Further, in FIG. 4, T2 indicates a second maximum value timing at which the position of the waist of the user in the z-axis direction becomes a maximum value next to the first maximum value timing T1 in the temporal transition of the position of the waist of the user in the z-axis direction. The first maximum value timing is an example of a first timing, and the second maximum value timing is an example of a second timing. Regardless of whether the running action of the user is the first running action or the second running action, a takeoff timing Ts is included in a time section from the first maximum value timing T1 to the second maximum value timing T2.

When the running action of the user is the second running action, the takeoff timing Ts is later and time from the takeoff timing Ts to the second maximum value timing T2 is shorter than when the running action of the user is the first running action. In other words, the time from the first maximum value timing T1 to the takeoff timing Ts is longer when the running action of the user is the second running action than when the running action of the user is the first running action. Thus, a reference ratio as a ratio of time D2 from the first maximum value timing T1 until the takeoff timing Ts to time D1 from the first maximum value timing T1 until the second maximum value timing T2 is higher when the running action of the user is the second running action than when the running action of the user is the first running action. The reference ratio corresponding to the takeoff timing Ts is expressed by the following equation (1). In the equation (1), R represents the reference ratio corresponding to the takeoff timing Ts, D1 represents the time from the first maximum value timing T1 to the second maximum value timing T2, and D2 represents the time from the first maximum value timing T1 to the takeoff timing Ts.

R=D2/D1 (1)

In FIG. 3 again, the running action identifier 202 identifies whether the running action of the user is the first running action or the second running action in accordance with a magnitude relationship between the reference ratio corresponding to the takeoff timing and a predetermined identification threshold value. That is, when the reference ratio corresponding to the takeoff timing is less than the identification threshold value, the running action identifier 202 identifies the running action of the user as the first running action, and when the reference ratio corresponding to the takeoff timing is equal to or more than the identification threshold value, the running action identifier identifies the running action of the user as the second running action. By an experiment in which a plurality of humans other than the user of the detection device 2 is designated as subjects, the identification threshold value is preset in accordance with a correlation between a type of the running action of the subjects and the reference ratio obtained by measuring the reference ratio in a case where the running action of the subjects is the first running action and the reference ratio in a case where the running action of the subjects is the second running action. In this embodiment, the identification threshold value is set to 0.95. Note that, in the experiment described above, the reference ratio only has to be measured by causing the subject to perform the running action on a force plate that detects the takeoff action by the subject with the acceleration sensor (not illustrated) attached to the waist of the subject, detecting the takeoff timing in accordance with the detection result of the takeoff action by the force plate, and detecting the first maximum value timing and the second maximum value timing in accordance with the temporal transition of the position of the waist of the subject obtained by integrating an output signal of the acceleration sensor twice. Note that, it has been described that a plurality of humans other than the user of the detection device 2 is designated as subjects in the experiment in this embodiment, but this is merely an example. In the experiment, the user of the detection device 2 may be the subject of the experiment, or a plurality of humans including the user of the detection device 2 and one or more humans other than the user of the detection device 2 may be the subject.

The running action identifier 202 causes a takeoff type estimator stored in the estimator storage 203 to estimate whether the reference ratio corresponding to the takeoff timing is less than the identification threshold value in accordance with the acceleration data acquired by the acceleration data acquirer 200, and identifies whether the running action of the user is the first running action or the second running action in accordance with a result of the estimation. That is, the running action identifier 202 identifies the running action of the user as the first running action when the takeoff type estimator estimates that the reference ratio according to the takeoff timing is less than the identification threshold value, and identifies the running action of the user as the second running action when the takeoff type estimator estimates that the reference ratio according to the takeoff timing is equal to or more than the identification threshold value.

The takeoff type estimator is a support vector machine (SVM), and estimates whether the reference ratio corresponding to the takeoff timing is less than the identification threshold value in accordance with the acceleration data. Specifically, in response to input of the acceleration of the user in the x-axis direction, the y-axis direction, and the z-axis direction as a feature amount at the timing before the second maximum value timing indicated by the acceleration data by a predetermined first reference time (in this embodiment, 500 ms), the takeoff type estimator estimates whether the reference ratio corresponding to the takeoff timing is less than the identification threshold value. The takeoff type estimator is created by an information processor 4 to be described later, then taken into the detection device 2, and stored in advance in the estimator storage 203. The creation of the takeoff type estimator by the information processor 4 will be described later. Note that, in the description of this embodiment, the takeoff type estimator is a support vector machine, but this is merely an example. The takeoff type estimator may be any estimator that estimates whether the reference ratio is less than the identification threshold value. For example, the takeoff type estimator may be a deep neural network (DNN).

When performing the estimation with use of the takeoff type estimator, the running action identifier 202 detects the first maximum value timing and the second maximum value timing in accordance with the acceleration data acquired by the acceleration data acquirer 200, inputs the acceleration of the user in the x-axis direction, the y-axis direction, and the z-axis direction at the timing before the detected second maximum value timing indicated by the acceleration data by the first reference time to the takeoff type estimator as the feature amount, and thus causes the takeoff type estimator to estimate whether the reference ratio according to the takeoff timing is less than the identification threshold value. Specifically, the running action identifier 202 acquires the z-axis position data indicating the temporal transition of the position of the waist of the user in the z-axis direction by integrating the z-axis acceleration data included in the acceleration data acquired by the acceleration data acquirer 200 twice. As described above, since the acceleration data is acquired in accordance with the acceleration signal output from the acceleration sensor 28a built in the detection device 2 attached to the waist of the user, the z-axis position data can be acquired by integrating the z-axis acceleration data included in the acceleration data twice. The running action identifier 202 performs smoothing processing using a moving average filter on the acquired z-axis position data, and detects the first maximum value timing and the second maximum value timing in accordance with the z-axis position data subjected to the smoothing processing. In the description of this embodiment, the running action identifier 202 performs the smoothing processing with use of the moving average filter, but this is merely an example. The running action identifier 202 can perform the smoothing processing on the z-axis position data with use of any smoothing filter such as a low-pass filter or a Gaussian filter.

When the running action of the user is the first running action, the timing detector 204 detects the takeoff timing by a first detection method, and when the running action of the user is the second running action, the timing detector detects the takeoff timing by a second detection method different from the first detection method. Specifically, the timing detector 204 detects the takeoff timing by the first detection method when the running action identifier 202 identifies the running action of the user as the first running action, and detects the takeoff timing by the second detection method when the running action identifier 202 identifies the running action of the user as the second running action.

Whether the timing detector 204 detects the takeoff timing using the first detection method or the second detection method, the timing detector detects the takeoff timing in accordance with the temporal transition of the acceleration of the user in a detection target direction and a template stored by the template storage 205, the temporal transition being indicated by the acceleration data acquired by the acceleration data acquirer 200. The template is data that indicates the temporal transition of the acceleration of the subject when the subject performs the takeoff action. Specifically, the template includes data of the plurality of sampling points in chronological order, and the data of one sampling point included in the template indicates the acceleration of the subject detected at the one sampling point. The template includes data that indicates the timing at which the subject performs the takeoff action. The template is created by the information processor 4 to be described later, then taken into the detection device 2, and stored in advance in the template storage 205. The creation of the template by the information processor 4 will be described later. The template is an example of reference data.

The detection target direction is different between when the timing detector 204 detects the takeoff timing by the first detection method and when the timing detector 204 detects the takeoff timing by the second detection method. Specifically, in the first detection method, the y-axis direction is set as the detection target direction, and the takeoff timing is detected in accordance with the temporal transition of the acceleration of the user in the y-axis direction, the temporal transition being indicated by the acceleration data. On the other hand, in the second detection method, the z-axis direction is set as the detection target, and the takeoff timing is detected in accordance with the temporal transition of the acceleration of the user in the z-axis direction, the temporal transition being indicated by the acceleration data. Further, the template used for the detection is different between when the timing detector 204 detects the takeoff timing by the first detection method and when the timing detector 204 detects the takeoff timing by the second detection method. Specifically, the template storage 205 stores a y-axis template and a z-axis template as templates. The y-axis template is a template that indicates the temporal transition of the acceleration of the subject in the y-axis direction when the subject performs the takeoff action. The z-axis template is a template that indicates the temporal transition of the acceleration of the subject in the z-axis direction when the subject performs the takeoff action. The y-axis template and the z-axis template each include data indicating the timing at which the subject performs the takeoff action. The y-axis template corresponds to the y-axis direction, and the z-axis template corresponds to the z-axis direction. In the first detection method, the takeoff timing is detected with use of the y-axis template corresponding to the y-axis direction as the detection target direction in the first detection method. In the second detection method, the takeoff timing is detected with use of the z-axis template corresponding to the z-axis direction as the detection target direction in the second detection method. The y-axis template is an example of first reference data, and the z-axis template is an example of second reference data.

It has been found from the experiment performed by the inventor(s) of the present invention that, in a case where the user performs the takeoff action when the user is moving by performing the first running action, a characteristic transition waveform of the acceleration when the takeoff action is performed appears more remarkably in the temporal transition of the acceleration of the user in the y-axis direction than in the temporal transition of the acceleration of the user in the z-axis direction. On the other hand, it has been found from the experiment performed by the inventor(s) of the present invention that, in a case where the user performs the takeoff action when the user is moving by performing the second running action, a characteristic transition waveform of the acceleration when the takeoff action is performed appears more remarkably in the temporal transition of the acceleration of the user in the z-axis direction than in the temporal transition of the acceleration of the user in the y-axis direction. In a case where the running action of the user is the first running action, the timing detector 204 detects the takeoff timing with use of the y-axis template corresponding to the y-axis direction in accordance with the temporal transition of the acceleration of the user in the y-axis direction, the temporal transition being indicated by the acceleration data by the first detection method. In a case where the running action of the user is the second running action, the timing detector detects the takeoff timing with use of the z-axis template corresponding to the z-axis direction in accordance with the temporal transition of the acceleration of the user in the z-axis direction, the temporal transition being indicated by the acceleration data by the second detection method. Thus, detection accuracy of the takeoff timing is improved. In particular, this configuration can improve the detection accuracy of the takeoff timing when the running action of the user is the second running action, that is, when the user is running at a low speed. That is, the timing detector 204 detects the takeoff timing by a different detection method depending on which of the first running action or the second running action the running action of the user is, therefore detects the takeoff timing by an appropriate detection method in accordance with the type of the running action of the user, and improves the detection accuracy of the takeoff timing.

Whether the first detection method or the second detection method is used to detect the takeoff timing, the timing detector 204 associates acceleration data corresponding to the detection target direction among the x-axis acceleration data, the y-axis acceleration data, and the z-axis acceleration data included in the acceleration data with a template corresponding to the detection target direction among the y-axis template and the z-axis template stored by the template storage 205 by a dynamic time warping (DTW) method, and detects, as the takeoff timing, a timing in the acceleration data corresponding to a timing indicated by the template at which the subject performs the takeoff action That is, in the case where the takeoff timing is detected by the first detection method, the timing detector 204 associates the y-axis acceleration data corresponding to the y-axis direction as the detection target direction in the first detection method with the y-axis template corresponding to the y-axis direction by the dynamic time warping method, and detects, as the takeoff timing, the timing in the y-axis acceleration data corresponding to the timing indicated by the y-axis template at which the subject performs the takeoff action. In the case where the takeoff timing is detected by the second detection method, the timing detector 204 associates the z-axis acceleration data corresponding to the z-axis direction as the detection target direction in the second detection method with the z-axis template corresponding to the z-axis direction by the dynamic time warping method, and detects, as the takeoff timing, the timing in the z-axis acceleration data corresponding to the timing indicated by the z-axis template at which the subject performs the takeoff action.

Specifically, the timing detector 204 associates the data included in a search section among the acceleration data corresponding to the detection target direction with the template corresponding to the detection target direction by the dynamic time warping method, and detects, as the takeoff timing, the timing in the acceleration data corresponding to the timing indicated by the template at which the subject performs the takeoff action. The search section is a time section corresponding to the first maximum value timing and the second maximum value timing described above. Specifically, the search section is a time section from a timing before the second maximum value timing by a second reference time to the second maximum value timing, and the second reference time is time obtained by multiplying time from the first maximum value timing to the second maximum value timing by a predetermined reference multiplier (in this embodiment, 0.5).

By limiting the acceleration data associated with the template by the dynamic time warping method to the acceleration data included in the search section described above, a calculation load can be reduced. There is a low possibility that the takeoff timing to be detected is included in a section other than the search section, and even if the detection section of the takeoff timing is limited to the search section, the detection accuracy of the takeoff timing hardly decreases. On the contrary, in a case where a section other than the search section is included in the detection section of the takeoff timing, a timing that is included in the section other than the search section and at which the user does not actually perform the takeoff action is erroneously detected as the takeoff timing, and thus, there is a possibility that the detection accuracy of the takeoff timing is lowered. Therefore, the timing detector 204 detects the takeoff timing by associating the acceleration data with the template included in the search section by the dynamic time warping method, and this improves the detection accuracy of the takeoff timing while reducing the calculation load.

A time length of the search section varies depending on a running speed of the user. For this reason, the time length of the acceleration data associated with the template varies due to variation in the running speed of the user. The timing detector 204 associates the acceleration data with the template by the dynamic time warping method, can thus appropriately associate the acceleration data with the template regardless of the time length of the acceleration data. Therefore, by the dynamic time warping method, the timing detector 204 can appropriately associate the acceleration data with the template and detect the takeoff timing regardless of the variation in the time length of the acceleration data caused by the variation in the running speed of the user. In other words, the timing detector 204 detects the takeoff timing by associating the acceleration data with the template by the dynamic time warping method, and thus can detect the takeoff timing robustly against the variation in the running speed of the user and improve the detection accuracy of the takeoff timing.

When associating the acceleration data with the template by the dynamic time warping method, the timing detector 204 calculates a distance between the data of each sampling point of the acceleration data and the data of each sampling point of the template, and thus creates a distance matrix that indicates the distance between the data of each sampling point of the acceleration data and the data of each sampling point of the template. When creating the distance matrix, the timing detector 204 calculates an absolute value of a difference between the data as the distance between the data. In the dynamic time warping method, the distance between the data may be referred to as cost. In this embodiment, the timing detector 204 calculates the absolute value of the difference between the data as the distance between the data, but this is merely an example, and the timing detector 204 can calculate any distance as the distance between the data. For example, the timing detector 204 may calculate a cosine distance between the data as the distance between the data.

After creating the distance matrix described above, the timing detector 204 obtains a best path that is a path on the distance matrix and in which a sum of elements of the distance matrix on the path is smallest among paths in which an element of the distance matrix indicating the distance between data at a starting end of the acceleration data and data at a starting end of the template is set as a starting end and an element of the distance matrix indicating the distance between data at a terminal end of the acceleration data and data at a terminal end of the template is set as a terminal end. In the dynamic time warping method, the best path may also be referred to as warping path. The best path is a path that passes through a plurality of the elements of the distance matrix, in other words, a path that connects the plurality of elements. The best path is a path that is not interrupted on the way from the starting end to the terminal end, and is a path that does not go backward on the way from the starting end to the terminal end.

The best path indicates a correspondence relationship between the acceleration data and the template obtained by the dynamic time warping method. Specifically, when an element that indicates the distance between the data at the first sampling point of the acceleration data and the data at the second sampling point of the template is included as an element on the best path, these pieces of data correspond to each other, and the first sampling point and the second sampling point correspond to each other. The timing detector 204 associates the acceleration data with the template by obtaining the best path indicating the correspondence relationship between the acceleration data and the template by the dynamic time warping method. The data of each sampling point of the acceleration data is associated with the data of each sampling point of the template by the best path indicating the correspondence relationship between the acceleration data and the template. In other words, each sampling point of the acceleration data is associated with each sampling point of the template by the best path indicating the correspondence relationship between the acceleration data and the template.

The timing detector 204 obtains the best path indicating the correspondence relationship between the acceleration data and the template by the dynamic time warping method to associate the acceleration data with the template, and detects, as the takeoff timing, the timing in the acceleration data corresponding to the timing indicated by the template at which the user performs the takeoff action. That is, in a case where the element indicating the distance between the data of the timing indicated by the template at which the user performs the takeoff action and the data of one timing of the data of each sampling point included in the acceleration data is included in the data of each sampling point included in the template as the obtained element on the best path, the timing detector 204 detects, as the takeoff timing, the one timing corresponding to the timing at which the user performs the takeoff action. In this case, among the data of each sampling point included in the template, the data at the timing indicated by the template at which the user performs the takeoff action corresponds to the data at one timing among the data of each sampling point included in the acceleration data.

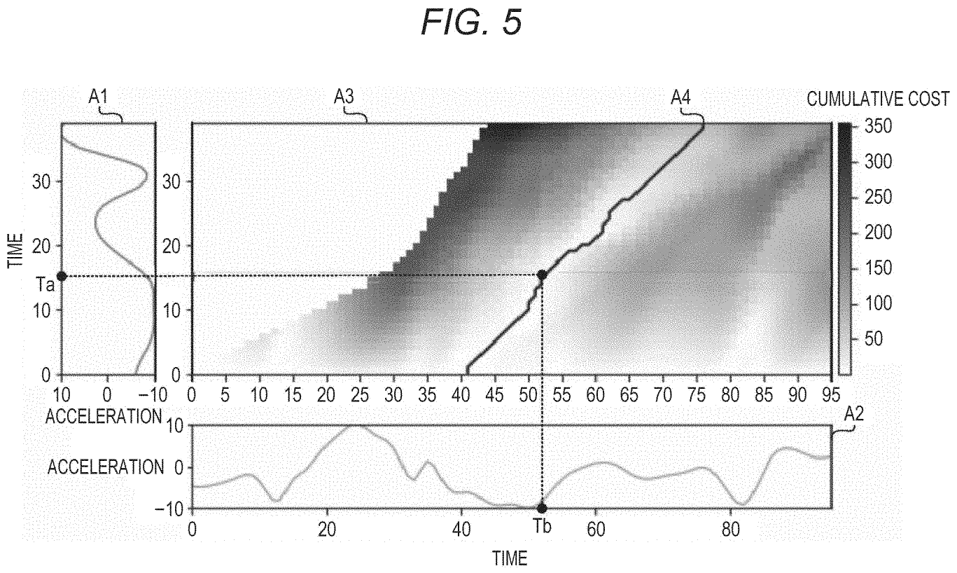

Hereinafter, the correspondence relationship between the template and the acceleration data will be specifically described with reference to FIG. 5. FIG. 5 illustrates a cumulative cost map A3 that represents a correspondence relationship between a y-axis template A1 and y-axis acceleration data A2. The y-axis acceleration data A2 is y-axis acceleration data corresponding to the time section from the first maximum value timing to the second maximum value timing described above, and is normalized in a range from 0 to 100 in a time axis direction, and the acceleration is normalized in a range from −10 to 10. A vertical axis of the cumulative cost map A3 is associated with a time axis of the y-axis template A1, and a horizontal axis of the cumulative cost map A3 is associated with a time axis of the y-axis acceleration data A2.

A cell in the cumulative cost map A3 is colored in darker gray as a cumulative cost corresponding to the cell is larger. The cumulative cost corresponding to the cell is a sum of the elements of the distance matrix indicating the distance between the data of each sampling point of the y-axis template A1 and the data of each sampling point of the y-axis acceleration data A2 on a best path to the cell. The best path to the cell is the path on the distance matrix described above. A starting end of the best path to the cell is an element of the above-described distance matrix indicating a distance between data at a starting end of the y-axis template A1 and data at a starting end of the y-axis acceleration data A2. A terminal end of the best path to the cell is an element of the distance matrix indicating the distance between the data of sampling timing corresponding to the cell of the y-axis template A1 and the data of sampling timing corresponding to the cell of the y-axis acceleration data A2. The best path to the cell is a path in which the sum of the elements of the distance matrix on the path is smallest among the paths on the distance matrix from the starting end to the terminal end. Among data of a plurality of the sampling timings included in the y-axis template A1, the data of the sampling timing on the time axis of the y-axis template A1 corresponding to a position in a vertical axis direction of the cumulative cost map A3 of one cell in the cumulative cost map A3 is the data of the sampling point corresponding to the one cell of the y-axis template A1. Further, among data of a plurality of the sampling timings included in the y-axis acceleration data A2, the data of the sampling timing on the time axis of the y-axis acceleration data A2 corresponding to a position in the horizontal axis direction of the cumulative cost map A3 of one cell in the cumulative cost map A3 is the data of the sampling point corresponding to the one cell of the y-axis acceleration data A2.

In the cumulative cost map A3, the cumulative cost corresponding to each cell is illustrated, and a best path A4 indicating the correspondence relationship between the y-axis template A1 and the y-axis acceleration data A2 is illustrated. The data of each sampling point of the y-axis template A1 is associated with the data of each sampling point of the y-axis acceleration data A2 by the best path A4. In other words, each sampling point of the y-axis template A1 is associated with each sampling point of the y-axis acceleration data A2 by the best path A4. For example, as illustrated in FIG. 5, a timing Ta indicated by the y-axis template A1 at which the subject performs the takeoff action is associated with a timing Tb on the time axis of the y-axis acceleration data A2 by the best path A4. That is, an element indicating a distance between data of the timing Ta of the y-axis template A1 and data of the timing Tb of the y-axis acceleration data A2 is included as an element of the distance matrix on the best path A4, and these pieces of data are associated with each other by the best path A4. When the timing detector 204 detects the takeoff timing with use of the y-axis template A1 and the y-axis acceleration data A2 illustrated in FIG. 5, the timing Tb in the y-axis acceleration data A2 corresponding to the timing Ta indicated by the y-axis template A1 at which the subject performs the takeoff action is detected as the takeoff timing.

In FIG. 3 again, whether the first detection method or the second detection method is used to detect the takeoff timing, the timing detector 204 uses a prefilter stored in the prefilter storage 206 to perform prefilter processing of emphasizing the transition waveform of the characteristic acceleration when the user performs the takeoff action on the acceleration data corresponding to the detection target direction, associates the acceleration data subjected to the prefilter processing with the template corresponding to the detection target direction by the dynamic time warping method, and thus detects the takeoff timing. This configuration can improve the detection accuracy of the takeoff timing. The prefilter is created by the information processor 4 to be described later, then taken into the detection device 2, and stored in advance in the prefilter storage 206. The creation of the prefilter by the information processor 4 will be described later.

The prefilter used in the prefilter processing is different between when the timing detector 204 detects the takeoff timing by the first detection method and when the timing detector 204 detects the takeoff timing by the second detection method. Specifically, the prefilter storage 206 stores a y-axis prefilter and a z-axis prefilter as prefilters. The y-axis prefilter is a prefilter that emphasizes a characteristic transition waveform of the acceleration in the y-axis direction when the user performs the takeoff action. The z-axis prefilter is a prefilter that emphasizes a characteristic transition waveform of the acceleration in the z-axis direction when the user performs the takeoff action. In the first detection method, the y-axis acceleration data corresponding to the y-axis direction as the detection target direction in the first detection method is subjected to the prefilter processing with use of the y-axis prefilter corresponding to the y-axis direction, and the takeoff timing is detected in accordance with the y-axis acceleration data after being subjected to the prefilter processing. In the second detection method, the z-axis acceleration data corresponding to the z-axis direction as the detection target direction in the second detection method is subjected to the prefilter processing with use of the z-axis prefilter corresponding to the z-axis direction, and the takeoff timing is detected in accordance with the z-axis acceleration data after being subjected to the prefilter processing. This configuration can improve the detection accuracy of the takeoff timing by performing the prefilter processing with use of an appropriate prefilter in accordance with the type of the running action of the user.

In the description of this embodiment, the timing detector 204 performs the prefilter processing on the acceleration data corresponding to the detection target direction and detects the takeoff timing in accordance with the acceleration data corresponding to the detection target direction after the pre-filter processing is performed, but this is merely an example. The timing detector 204 may detect the takeoff timing in accordance with the acceleration data corresponding to the detection target direction and not subjected to the prefilter processing without performing the pre-filter processing. This configuration can reduce the calculation load.

In the description of this embodiment, the timing detector 204 detects the takeoff timing at which the takeoff action is performed, but this is merely an example. The timing detector 204 can detect a timing at which the moving user performs any action. For example, the timing detector 204 may detect a landing timing at which the user performs the landing action when the user is running (or walking). The landing action is an action in which the user who is running (or walking) lands a foot that has been away from the ground, on the ground. The landing action is an example of the target action, and the landing timing is an example of the target timing. In this case, the timing detector 204 can detect the landing timing by applying the above-described method of detecting the takeoff timing to the detection of the landing timing. Specifically, in this case, data indicating the temporal transition of the acceleration of the subject when the subject performs the landing action is stored in the template storage 205 in advance as a template. The template includes data that indicates the timing at which the subject performs the landing action. The timing detector 204 associates the acceleration data corresponding to the detection target direction with the template stored by the template storage 205 by the dynamic time warping method, and detects, as the landing timing, a timing in the acceleration data corresponding to the timing indicated by the template at which the subject performs the landing action. This configuration can improve detection accuracy of the landing timing.

The takeoff timing information storage 207 stores takeoff timing information that indicates a detection history of the takeoff timing by the timing detector 204. The takeoff timing information stores data that indicates the takeoff timing detected by the timing detector 204 in chronological order. Upon each detection of the takeoff timing, the timing detector 204 adds data that indicates the detected takeoff timing to the takeoff timing information stored in the takeoff timing information storage 207 to update the takeoff timing information. This configuration allows a detection result of the takeoff timing by the timing detector 204 to be accumulated in the takeoff timing information storage 207.

The running action information acquirer 208 acquires the running action information indicating the feature of the running action of the user in accordance with the takeoff timing detected by the timing detector 204. In this embodiment, the running action information acquirer 208 acquires, as the running action information, a plurality of takeoff timing intervals as time intervals of two takeoff timings adjacent to each other on the time axis, and acquires information that indicates an average takeoff interval as an average value of the acquired plurality of takeoff timing intervals. The user can improve the running speed by adjusting a manner of running of the user so as to reduce the average takeoff interval indicated by the running action information presented by the terminal device 3. Every time the timing detector 204 detects the takeoff timing and the takeoff timing information stored in the takeoff timing information storage 207 is updated in accordance with the detected takeoff timing, the running action information acquirer 208 acquires (that is, calculates) the average takeoff interval in accordance with the updated takeoff timing information, and thus acquires the running action information indicating the average takeoff interval.

In the description of this embodiment, the running action information acquirer 208 acquires the information indicating the average takeoff interval as the running action information, but this is merely an example. The running action information acquirer 208 can acquire any information that indicates the feature of the running action of the user as the running action information. For example, the timing detector 204 may be configured to detect the takeoff timing and the landing timing, and the running action information acquirer 208 may acquire, as the running action information, information that indicates a contact time as time during when at least one of the feet of the user is in contact with the ground in accordance with the takeoff timing and the landing timing detected by the timing detector 204. Specifically, the running action information acquirer 208 may acquire the time from the landing timing detected by the timing detector 204 to the takeoff timing detected by the timing detector 204 as the contact time. Alternatively, the running action information acquirer 208 may acquire, as the running action information, information that indicates an air stay time during which at least one of the feet of the user is away from the ground in accordance with the takeoff timing and the landing timing detected by the timing detector 204. Specifically, the running action information acquirer 208 only has to acquire the time from the takeoff timing detected by the timing detector 204 to the landing timing detected by the timing detector 204 as the air stay time.

The running action information output unit 209 outputs the running action information acquired by the running action information acquirer 208. Specifically, the running action information output unit 209 controls the communicator 25 to transmit the running action information to the terminal device 3. In the description of this embodiment, the running action information output unit 209 transmits the running action information to the terminal device 3, but this is merely an example. The running action information output unit 209 can output the running action information by any method. For example, the running action information output unit 209 may present the running action information to the user by outputting the running action information in a form that is recognizable by the user. Specifically, the running action information output unit 209 may present the running action information to the user by causing the above-described display device to display an image corresponding to the running action information.

As described above, the timing detector 204 improves the detection accuracy of the takeoff timing by detecting the takeoff timing by the detection method corresponding to the type of the running action of the user. Further, as described above, the running action information acquirer 208 acquires the running action information in accordance with the takeoff timing detected by the timing detector 204, and the running action information output unit 209 outputs the running action information acquired by the running action information acquirer 208. This configuration can improve the accuracy of the running action information and convenience for the user.

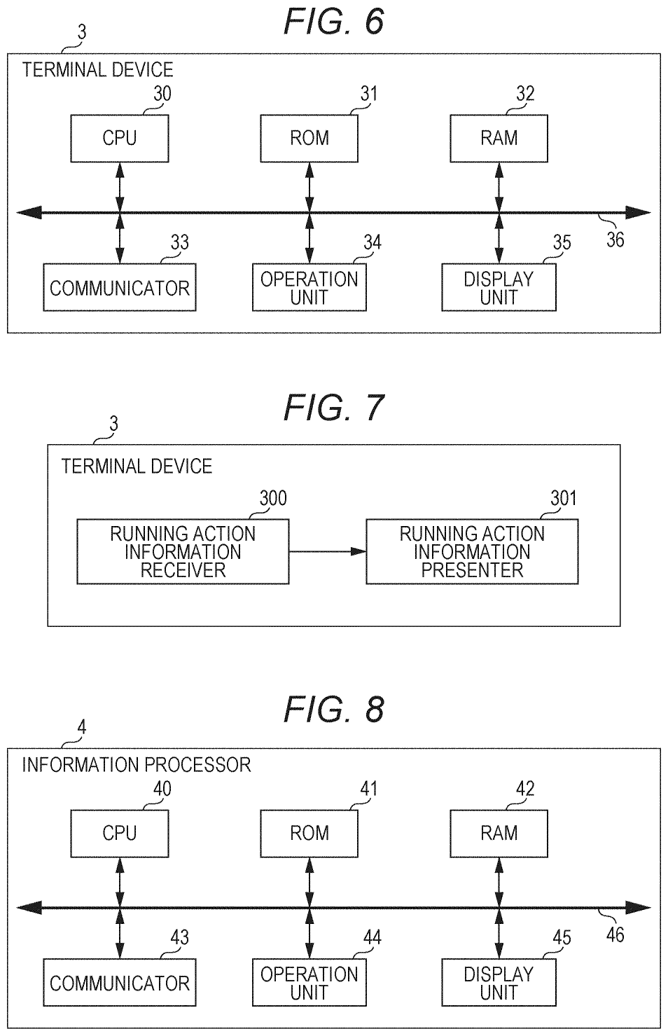

Hereinafter, physical and functional configurations of the terminal device 3 will be described with reference to FIGS. 6 and 7. First, the physical configuration of the terminal device 3 will be described with reference to FIG. 6. As illustrated in FIG. 6, the terminal device 3 includes a CPU 30, a ROM 31, a RAM 32, a communicator 33, an operation unit 34, and a display unit 35. The CPU 30 to the display unit 35 are interconnected via a system bus 36 as a command and data transmission path. Note that FIG. 6 illustrates only a configuration related to a characteristic part of the present invention in the physical configuration included in the terminal device 3. The terminal device 3 may include any physical configuration not shown in FIG. 6. For example, the terminal device 3 may include a speaker that outputs a voice or a microphone that records a voice uttered by the user.

The CPU 30 controls each unit of the terminal device 3 in accordance with a program and data stored in the ROM 31 and executes various processing. The ROM 31 stores, in a non-transitory manner, a program and data used by the CPU 30 to execute various processing. The RAM 32 functions as a work area of the CPU 30. That is, the CPU 30 reads the program and data stored in the ROM 31 to the RAM 32, and executes various processing by referring to the read program and data. In addition, the CPU 30 temporarily stores data acquired by executing various processing in the RAM 32, and executes various processing by referring to the stored data. The communicator 33 performs wireless communication or wired communication with an external device including the detection device 2 under the control of the CPU 30, and transmits and receives data. The communicator 33 outputs the data received from the external device to the CPU 30. The operation unit 34 includes a touch screen that receives a contact operation by the user, detects a contact operation on the touch screen by the user, and outputs a contact operation signal that indicates a detection result to the CPU 30. By performing a contact operation on the touch screen included in the operation unit 34, the user can input various instructions to the terminal device 3, including an instruction to the terminal device 3 to start presentation of the running action information and an instruction to the terminal device 3 to end the presentation of the running action information. Further, the operation unit 34 includes a plurality of keys that receives an operation by the user, including a terminal device power key that receives an operation of switching on and off a power source of the terminal device 3, detects the operation on the plurality of keys by the user, and outputs a key operation signal that indicates a detection result to the CPU 30. The display unit 35 includes a display such as a liquid crystal display or an organic electroluminescence (EL) display, and displays various images under the control of the CPU 30. The touch screen included in the operation unit 34 is disposed to be superimposed on the display included in the display unit 35.

The terminal device 3 having the above-described physical configuration functionally includes a running action information receiver 300 and a running action information presenter 301 as illustrated in FIG. 7. The running action information receiver 300 and the running action information presenter 301 are realized by the CPU 30. That is, the CPU 30 executes the program stored in the ROM 31 to control each unit of the terminal device 3, and thus functions as the running action information receiver 300 and the running action information presenter 301. Note that FIG. 7 illustrates only a functional configuration related to a characteristic part of the present invention in functional configuration included in the terminal device 3. The terminal device 3 may have any functional configuration not shown in FIG. 7. For example, the terminal device 3 may include a voice output controller that controls voice output by the speaker described above.

The running action information receiver 300 receives the running action information from the detection device 2. Specifically, the running action information receiver 300 controls the communicator 33 to receive the running action information transmitted from the communicator 25 of the detection device 2 in accordance with the control by the running action information output unit 209 of the detection device 2 described above. The running action information presenter 301 presents the running action information received from the detection device 2 by the running action information receiver 300 to the user by outputting the running action information in a form recognizable by the user. Specifically, the running action information presenter 301 presents the running action information to the user by controlling the display unit 35 to display a running action information image corresponding to the received running action information. Specifically, the running action information presenter 301 controls the display unit 35 to display, as the running action information image, an image that indicates the average takeoff interval indicated by the received running action information (for example, an image representing a message “the current average takeoff interval is X seconds”). When the running action information receiver 300 newly receives the running action information when the display unit 35 is already displaying the running action information image, the running action information presenter 301 controls the display unit 35 to update the displayed running action information image in accordance with the newly received running action information.

In the description of this embodiment, the running action information presenter 301 presents the running action information to the user by causing the display unit 35 to display the running action information image corresponding to the running action information, but this is merely an example. The running action information presenter 301 can present the running action information to the user by outputting the running action information in any form recognizable by the user. For example, the running action information presenter 301 may present the running action information to the user by causing the speaker to output a running action information voice corresponding to the running action information (for example, a voice representing a message “the current average takeoff interval is X seconds”).

Hereinafter, the creation of the takeoff type estimator, prefilter, and template will be described. In this embodiment, the takeoff type estimator, the prefilter, and the template are created by the external information processor 4 illustrated in FIG. 8 and then taken into the detection device 2.

The information processor 4 is an information processing device such as a computer operated by an operator in a factory manufacturing the detection device 2, and includes a CPU 40, a ROM 41, a RAM 42, a communicator 43, an operation unit 44, and a display unit 45 as illustrated in FIG. 8. The operator described above is an operating staff who operates the information processor 4. The CPU 40 to the display unit 45 are interconnected via a system bus 46 as a command and data transmission path. The CPU 40 controls each unit of the information processor 4 in accordance with a program and data stored in the ROM 41, and executes various processing. The ROM 41 stores, in a non-transitory manner, a program and data used by the CPU 40 to execute various processing. The RAM 42 functions as a work area of the CPU 40. That is, the CPU 40 reads the program and data stored in the ROM 41 to the RAM 42, and executes various processing by referring to the read program and data. In addition, the CPU 40 temporarily stores data acquired by executing various processing in the RAM 42, and executes various processing by referring to the stored data. The communicator 43 performs wireless communication or wired communication with an external device including the detection device 2 under the control of the CPU 40, and transmits and receives data. The communicator 43 outputs the data received from the external device to the CPU 40. The operation unit 44 includes a keyboard, a mouse, and the like for receiving an operation by the operator, detects an operation on the keyboard, the mouse, and the like by the operator, and outputs an operation signal that indicates a detection result to the CPU 40. By performing an operation on the keyboard, the mouse, or the like, the operator can input various instructions to the information processor 4, including an instruction to the information processor 4 to create a takeoff type estimator, an instruction to the information processor 4 to create a prefilter, and an instruction to the information processor 4 to create a template. The display unit 45 includes a display such as a liquid crystal display or an organic EL display, and displays various images under the control of the CPU 40.

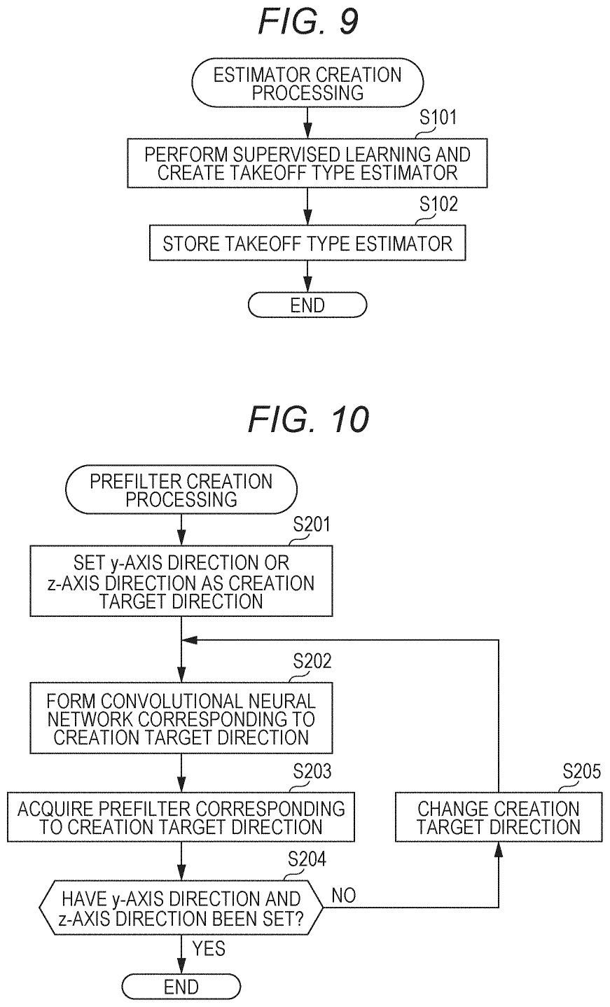

Hereinafter, the creation of the takeoff type estimator by the information processor 4 will be described. The information processor 4 executes estimator creation processing illustrated in a flowchart in FIG. 9 to create the takeoff type estimator. Hereinafter, the estimator creation processing illustrated in the flowchart in FIG. 9 will be described.

The ROM 41 of the information processor 4 stores in advance a plurality of pieces of estimator learning data used by the CPU 40 to create the takeoff type estimator. Specifically, the ROM 41 stores a predetermined first reference number (in this embodiment, 150) of pieces of estimator learning data. The plurality of pieces of estimator learning data stored in the ROM 41 includes a plurality of pieces of positive example estimator learning data and a plurality of pieces of negative example estimator learning data. The positive example estimator learning data is data that indicates the acceleration of the subject in the x-axis direction, the y-axis direction, and the z-axis direction at a timing before the second maximum value timing by the first reference time (in this embodiment, 500 ms) when the reference ratio corresponding to the takeoff timing is less than the identification threshold value. The negative example estimator learning data is data that indicates the acceleration of the subject in the x-axis direction, the y-axis direction, and the z-axis direction at a timing before the second maximum value timing by the first reference time when the reference ratio corresponding to the takeoff timing is equal to or more than the identification threshold value. A positive example class label is given in advance to the positive example estimator learning data, and a negative example class label is given in advance to the negative example estimator learning data.

In this embodiment, in an experiment in which a plurality of humans other than the user of the detection device 2 are designated as subjects, each subject performs the running action in a state where the acceleration sensor (not illustrated) is attached to the waist of the subject. Data that indicates the acceleration of the subject in the x-axis direction, the y-axis direction, and the z-axis direction at a timing before a second maximum value timing detected by the acceleration sensor by the first reference time is acquired as the estimator learning data and are stored in advance in the ROM 41. Specifically, in this embodiment, in the experiment described above, the subject performs the running action on a force plate, the takeoff timing is detected in accordance with a detection result of the takeoff action of each subject by the force plate, the first maximum value timing and the second maximum value timing are detected in accordance with the temporal transition of the position of the waist of the subject obtained by integrating the output signal of the acceleration sensor twice, and thus measures the reference ratio corresponding to the takeoff timing. Then, positive example estimator learning data is created by giving a positive example label to data that indicates the acceleration when the reference ratio is less than the identification threshold value, negative example estimator learning data is created by giving a negative example label to data that indicates the acceleration when the reference ratio is equal to or more than the identification threshold value, and the created positive example estimator learning data and negative example estimator learning data are stored in advance in the ROM 41 as the estimator learning data. Note that, it has been described that a plurality of humans other than the user of the detection device 2 is designated as subjects in the experiment in this embodiment, but this is merely an example. In the experiment, the user of the detection device 2 may be the subject of the experiment, or a plurality of humans including the user of the detection device 2 and one or more humans other than the user of the detection device 2 may be the subject.

In a state where the estimator learning data is stored in advance in the ROM 41, when the operator inputs an instruction to the information processor 4 to create the takeoff type estimator by operating the keyboard, the mouse, or the like included in the operation unit 44, the CPU 40 starts the estimator creation processing illustrated in the flowchart in FIG. 9. Upon start of the estimator creation processing, the CPU 40 first performs supervised learning using the estimator learning data stored in the ROM 41 as teacher data to create the takeoff type estimator (step S101). In the supervised learning performed in step S101, the acceleration of the subject at the timing before the second maximum value timing indicated by the estimator learning data by the first reference time is input as a feature amount to the takeoff type estimator, and is identified as a positive example or a negative example, and thus a takeoff type identifier is created. After execution of the processing of step S101, the CPU 40 stores the takeoff type estimator created in step S101 in the RAM 42 (step S102), and ends the estimator creation processing.

The detection device 2 communicates with the information processor 4 with use of the communicator 25 to take in, from the information processor 4, the takeoff type estimator created by the information processor 4 performing the estimator creation processing described above, and stores the takeoff type estimator in the estimator storage 203 provided in the storage area of the ROM 23.

Next, the creation of the prefilter by the information processor 4 will be described. The information processor 4 executes prefilter creation processing illustrated in a flowchart in FIG. 10 to create the prefilter. Hereinafter, the prefilter creation processing illustrated in the flowchart in FIG. 10 will be described.

In the ROM 41 of the information processor 4, a plurality of pieces of CNN learning data used by the CPU 40 for forming a convolutional neural network (CNN) to be described later is stored in advance. Specifically, the ROM 41 stores a predetermined second reference number (in this embodiment, 200) of pieces of CNN learning data. The plurality of pieces of CNN learning data stored in the ROM 41 includes a plurality of pieces of y-axis CNN learning data and a plurality of pieces of z-axis CNN learning data.

The y-axis CNN learning data is CNN learning data corresponding to the y-axis direction, and the z-axis CNN learning data is CNN learning data corresponding to the z-axis direction. The plurality of pieces of y-axis CNN learning data includes a plurality of pieces of y-axis takeoff data and a plurality of pieces of y-axis non-takeoff data. The y-axis takeoff data is data that indicates a temporal transition of the acceleration of the subject in the y-axis direction over a predetermined third reference time (in the present embodiment, 3.0 s) in a case where the subject is performing the takeoff action. A label indicating that the subject is perform the takeoff action is given in advance to the y-axis takeoff data. The y-axis non-takeoff data is data that indicates a temporal transition of the acceleration of the subject in the y-axis direction over the third reference time in a case where the subject is not performing the takeoff action. A label indicating that the subject is not performing the takeoff action is given in advance to the y-axis non-takeoff data.

The plurality of pieces of z-axis CNN learning data includes a plurality of pieces of z-axis takeoff data and a plurality of pieces of z-axis non-takeoff data. The z-axis takeoff data is data that indicates a temporal transition of the acceleration of the subject in the z-axis direction over the third reference time in the case where the subject is performing the takeoff action. A label indicating that the subject is performing the takeoff action is given in advance to the z-axis takeoff data. The z-axis non-takeoff data is data that indicates a temporal transition of the acceleration of the subject in the z-axis direction over the third reference time in the case where the subject is not performing the takeoff action. A label indicating that the subject is not performing the takeoff action is given in advance to the z-axis non-takeoff data.

In this embodiment, in an experiment in which a plurality of humans other than the user of the detection device 2 is designated as subjects, each subject performs the running action with the acceleration sensor (not illustrated) attached to the waist of the subject. The acceleration sensor acquires data that indicates the temporal transition of the acceleration of the subject in the y-axis direction and the z-axis direction. The y-axis takeoff data, the y-axis non-takeoff data, the z-axis takeoff data, and the z-axis non-takeoff data are acquired in accordance with the data, and are stored in advance in the ROM 41. Specifically, from the data indicating the temporal transition of the acceleration of the subject in the y-axis direction acquired by the acceleration sensor, the data corresponding to the time section of a length of the third reference time including the timing at which the subject performs the takeoff action is cut out. The label indicating that the takeoff action is performed is given to the cut data to create the y-axis takeoff data. From the data indicating the temporal transition of the acceleration of the subject in the y-axis direction acquired by the acceleration sensor, the data corresponding to the time section of a length of the third reference time not including the timing at which the subject performs the takeoff action is cut out. The label indicating that the takeoff action is not performed is given to the cut data to create the y-axis non-takeoff data.

From the data indicating the temporal transition of the acceleration of the subject in the z-axis direction acquired by the acceleration sensor, the data corresponding to the time section of a length of the third reference time including the timing at which the subject performs the takeoff action is cut out. The label indicating that the takeoff action is performed is given to the cut data to create the z-axis takeoff data. From the data indicating the temporal transition of the acceleration of the subject in the z-axis direction acquired by the acceleration sensor, the data corresponding to the time section of a length of the third reference time not including the timing at which the subject performs the takeoff action is cut out. The label indicating that the takeoff action is not performed is given to the cut data to create the z-axis non-takeoff data. Upon detection of the temporal transition of the acceleration of the subject by the acceleration sensor described above, the subject performs the running action on the force plate, the takeoff action of the subject is detected by the force plate, and thus the takeoff timing is detected. Note that, it has been described that a plurality of humans other than the user of the detection device 2 is designated as subjects in the experiment in this embodiment, but this is merely an example. In the experiment, the user of the detection device 2 may be the subject of the experiment, or a plurality of humans including the user of the detection device 2 and one or more humans other than the user of the detection device 2 may be the subject.

In a state where the CNN learning data is stored in advance in the ROM 41, when the operator inputs an instruction to the information processor 4 to create the prefilter by operating the keyboard, the mouse, or the like included in the operation unit 44, the CPU 40 starts the prefilter creation processing illustrated in the flowchart in FIG. 10.

Upon start of the prefilter creation processing, the CPU 40 first randomly selects one of the y-axis direction or the z-axis direction and sets the selected direction as a creation target direction (step S201). After execution of the processing of step S201, the CPU 40 forms the convolutional neural network corresponding to the creation target direction by performing supervised learning using, as teacher data, the CNN learning data corresponding to the creation target direction and stored in the ROM 41 (step S202).

In response to the input of the data indicating the temporal transition of the acceleration of the subject in the creation target direction over the third reference time, the convolutional neural network corresponding to the creation target direction and created in step S202 outputs a probability that the subject has performed the takeoff action. In a case where the y-axis direction is set as the creation target direction, in the processing of step S202, the supervised learning is performed with use of, as teacher data, y-axis CNN learning data including a plurality of pieces of y-axis takeoff data and a plurality of pieces of y-axis non-takeoff data as CNN learning data corresponding to the y-axis direction, and a convolutional neural network corresponding to the y-axis direction is formed. In response to the input of the data indicating the temporal transition of the acceleration of the subject in the y-axis direction over the third reference time, the formed convolutional neural network corresponding to the y-axis direction outputs the probability that the subject has performed the takeoff action.

On the other hand, in a case where the z-axis direction is set as the creation target direction, in the processing of step S202, the supervised learning is performed with use of, as teacher data, z-axis CNN learning data including a plurality of pieces of z-axis takeoff data and a plurality of pieces of z-axis non-takeoff data as CNN learning data corresponding to the z-axis direction, and a convolutional neural network corresponding to the z-axis direction is formed. In response to the input of the data indicating the temporal transition of the acceleration of the subject in the z-axis direction over the third reference time, the formed convolutional neural network corresponding to the z-axis direction outputs the probability that the subject has performed the takeoff action.

In the processing of step S202, as the convolutional neural network corresponding to the creation target direction, a one-dimensional convolutional neural network is formed, the network including an input layer that receives input of data indicating the temporal transition of the acceleration of the subject in the creation target direction over the third reference time, an output layer that outputs the probability that the subject has performed the takeoff action, and an intermediate layer provided between the input layer and the output layer. The intermediate layer described above includes a one-dimensional convolution layer that has a predetermined third reference number (in this embodiment, 20) of channels and performs convolution processing on acceleration data input to the input layer, a pooling layer that has a plurality of channels and performs pooling processing such as maximum pooling on the output value of the one-dimensional convolution layer, and a fully-connected layer that performs weighted addition of the output value of each channel of the pooling layer and outputs the result to the output layer. In the processing of step S202, the CNN learning data corresponding to the creation target direction is used as teacher data, and a weight of each channel included in the one-dimensional convolution layer is optimized by any method such as momentum stochastic gradient descent (MSGD).

After execution of the processing of step S202, the CPU 40 acquires, as the prefilter corresponding to the creation target direction, the convolution filter of one channel randomly selected from among the third reference number of channels of the one-dimensional convolution layer of the convolutional neural network corresponding to the creation target direction and formed in step S202, and stores the convolution filter in the RAM 42 (step S203). That is, in a case where the creation target direction is set to the y-axis direction, the convolution filter of one randomly selected channel among the third reference number of channels included in the one-dimensional convolution layer of the convolutional neural network corresponding to the y-axis direction and formed in step S202 is acquired as the y-axis prefilter as the prefilter corresponding to the y-axis direction in the processing of step S203. On the other hand, in a case where the creation target direction is set to the z-axis direction, the convolution filter of one randomly selected channel among the third reference number of channels included in the one-dimensional convolution layer of the convolutional neural network corresponding to the z-axis direction and formed in step S202 is acquired as the z-axis prefilter as the prefilter corresponding to the z-axis direction in the processing of step S203. In the processing of step S203, the data indicating the weight of the channel as a coefficient that defines the convolution filter of the selected channel is stored in the RAM 42 as the data representing the prefilter.

After execution of the processing of step S203, the CPU 40 determines whether both the y-axis direction and the z-axis direction have already been set as the creation target directions (step S204). Upon determination that one of the y-axis direction or the z-axis direction has not yet been set as the creation target direction (step S204; No), the CPU 40 sets a direction that has not yet been set as the creation target direction of the y-axis direction or the z-axis direction as the creation target direction (step S205), and the processing returns to step S202.

On the other hand, upon determination that both the y-axis direction and the z-axis direction have already been set as the creation target directions (step S204; Yes), the CPU 40 ends the prefilter creation processing. As described above, until determining that both the y-axis direction and the z-axis direction have already been set as the creation target directions (step S204; Yes), the CPU 40 repeatedly executes the processing of steps S202 to S204, and thus acquires both the y-axis prefilter corresponding to the y-axis direction and the z-axis prefilter corresponding to the z-axis direction.

The detection device 2 communicates with the information processor 4 with use of the communicator 25 to take in the y-axis prefilter and the z-axis prefilter created by the information processor 4 performing the above-described prefilter creation processing from the information processor 4, and stores the y-axis prefilter and the z-axis prefilter in the prefilter storage 206 provided in the storage area of the ROM 23. In this embodiment, the prefilter is described as one convolution filter among the plurality of channels included in the one-dimensional convolution layer of the convolutional neural network, but this is merely an example. The prefilter may be any filter that emphasizes a characteristic transition waveform of the acceleration of the user when the user performs the takeoff action.

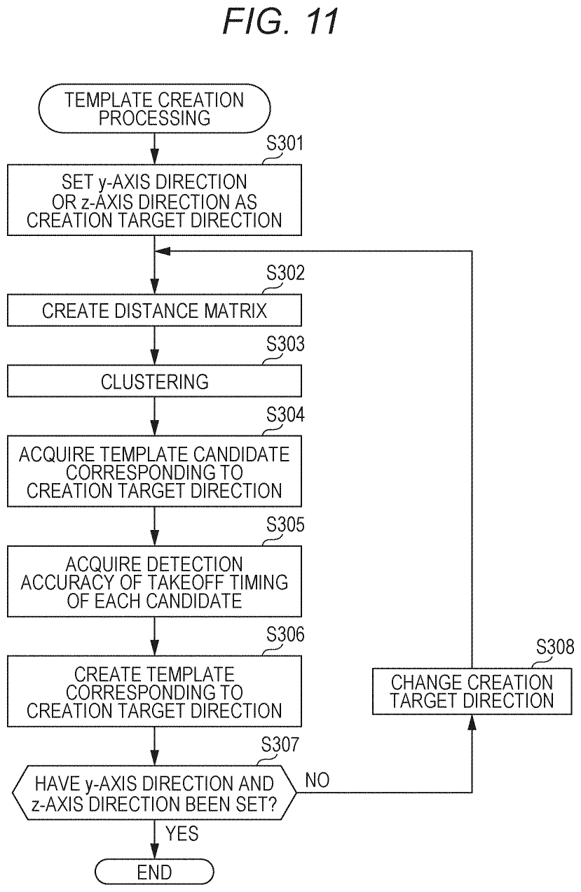

Next, the creation of the template by the information processor 4 will be described. The information processor 4 creates a template by executing template creation processing illustrated in a flowchart in FIG. 11. Hereinafter, the template creation processing illustrated in the flowchart in FIG. 11 will be described.

The ROM 41 of the information processor 4 stores in advance a plurality of pieces of template material data used by the CPU 40 to create a template. The plurality of pieces of template material data stored in the ROM 41 includes a predetermined fourth reference number (in this embodiment, 150) of pieces of y-axis template material data and the fourth reference number of pieces of z-axis template material data.

The y-axis template material data is template material data corresponding to the y-axis direction, and the z-axis template material data is template material data corresponding to the z-axis direction. The y-axis template material data indicates the temporal transition of the acceleration of the subject in the y-axis direction when the subject performs the takeoff action. Specifically, the y-axis template material data includes data of a plurality of sampling points in chronological order, and data of one sampling point included in the y-axis template material data indicates the acceleration of the subject in the y-axis direction detected at the one sampling point. The y-axis template material data includes in advance data that indicates the timing at which the subject performs the takeoff action. The z-axis template material data indicates the temporal transition of the acceleration of the subject in the z-axis direction when the subject performs the takeoff action. Specifically, the z-axis template material data includes data of a plurality of sampling points in chronological order, and data of one sampling point included in the z-axis template material data indicates the acceleration of the subject in the z-axis direction detected at the one sampling point. The z-axis template material data includes in advance data that indicates the timing at which the subject performs the takeoff action.

A method of creating the y-axis template material data will be described below. In this embodiment, in an experiment in which a plurality of humans other than the user of the detection device 2 are designated as subjects, the experiment is performed in which each subject performs the running action with the acceleration sensor (not illustrated) attached to the waist of the subject, and the y-axis template material data is created in accordance with data indicating the temporal transition of the acceleration of the subject in the y-axis direction detected by the acceleration sensor. Specifically, from the data indicating the temporal transition of the acceleration of the subject in the y-axis direction, the fourth reference number of pieces of data included in a time section between a timing at which one foot of the subject away from the ground contacts the ground and a timing at which, after the timing, the other foot of the subject in contact with the ground comes back in contact with the ground after leaving the ground is cut out. The timing at which the foot of the subject contacts the ground and the timing at which the foot of the subject leaves the ground are detected by causing the subject to perform the running action on the force plate and detecting the takeoff action and the landing action of the subject by the force plate in the above-described detection of the temporal transition of the acceleration of the subject by the acceleration sensor. Each of the cut fourth reference number of pieces of data is normalized such that a time length becomes an average value of time lengths of these pieces of data, and then resampled in the sampling frequency described above (in this embodiment, 200 Hz). Thereafter, from the fourth reference number of pieces of data after the resampling, the data included in a time section is cut out as the y-axis template material data, the time section having a starting end as a timing before a timing at which the subject performs the takeoff action by predetermined fourth reference time (in this embodiment, 15 ms) and having a terminal end as a timing after the timing at which the subject performs the takeoff action by the predetermined fifth reference time (in this embodiment, 25 ms). The z-axis template material data is created by applying a method in which the “y-axis direction” in the above method of creating the y-axis template material data is replaced with the “z-axis direction”. Note that, it has been described that a plurality of humans other than the user of the detection device 2 is designated as subjects in the experiment in this embodiment, but this is merely an example. In the experiment, the user of the detection device 2 may be the subject of the experiment, or a plurality of humans including the user of the detection device 2 and one or more humans other than the user of the detection device 2 may be the subject.

In the ROM 41, in addition to the template material data described above, test data used by the CPU 40 to evaluate a template candidate described later is stored in advance. The ROM 41 stores y-axis test data and z-axis test data as test data. The y-axis test data is test data corresponding to the y-axis direction, and the z-axis test data is test data corresponding to the z-axis direction. The y-axis test data is data indicating the temporal transition of the acceleration of the subject in the y-axis direction when the subject performs the takeoff action, and includes in advance data indicating the timing when the subject performs the takeoff action. The z-axis test data is data indicating the temporal transition of the acceleration of the subject in the z-axis direction when the subject performs the takeoff action, and includes in advance data indicating the timing when the subject performs the takeoff action. The y-axis test data and the z-axis test data are created in an experiment in which a plurality of humans other than the user of the detection device 2 are designated as subjects, the experiment is performed in which each subject performs the running action on the force plate with the acceleration sensor (not illustrated) attached to the waist of the subject, the temporal transition of the acceleration of the subject in the y-axis direction and the z-axis direction by the acceleration sensor, and the timing at which the subject performs the takeoff action by the force plate. Note that, it has been described that a plurality of humans other than the user of the detection device 2 is designated as subjects in the experiment in this embodiment, but this is merely an example. In the experiment, the user of the detection device 2 may be the subject of the experiment, or a plurality of humans including the user of the detection device 2 and one or more humans other than the user of the detection device 2 may be the subject.

In a state where the template material data and the test data are stored in advance in the ROM 41, when the operator inputs an instruction to the information processor 4 to create the template by operating the keyboard, the mouse, or the like included in the operation unit 44, the CPU 40 starts the template creation processing illustrated in the flowchart in FIG. 11.

Upon start of the template creation processing, the CPU 40 first randomly selects one of the y-axis direction or the z-axis direction and sets the selected direction as a creation target direction (step S301). After execution of the processing of step S301, the CPU 40 creates a distance matrix indicating a distance between the template material data corresponding to the creation target direction stored in the ROM 41 (step S302). In a case where the y-axis direction is set as the creation target direction, a distance matrix indicating a distance between the y-axis template material data corresponding to the y-axis direction is created in the processing of step S302. In a case where the z-axis direction is set as the creation target direction, a distance matrix indicating a distance between the z-axis template material data corresponding to the z-axis direction is created in the processing of step S302.

In the process of step S302, the CPU 40 creates a distance matrix by acquiring the distance between the template material data by the dynamic time warping method. Hereinafter, in the processing of step S302, the processing when the CPU 40 acquires the distance between one piece of template material data and another piece of template material data by the dynamic time warping method will be described. First, the CPU 40 creates a distance matrix indicating a distance between data of each sampling point of one piece of template material data and data of each sampling point of another piece of template material data. At this time, an absolute value of the difference between the data is calculated as a distance between the data. The CPU 40 obtains the best path indicating the correspondence relationship between one piece of template material data and another piece of template material data in accordance with the created distance matrix, and thus associates the one piece of template material data with the another piece of template material data. Then, the CPU 40 acquires a sum of the obtained elements on the best path among the elements of the distance matrix as a distance between one piece of template material data and another piece of template material data. In the processing of step S302, the CPU 40 repeatedly executes the same processing as the above processing to acquire the distance between each piece of the template material data corresponding to the creation target direction and all the other pieces of template material data corresponding to the creation target direction and create the distance matrix.

After execution of the processing of step S302, the CPU 40 classifies the template material data corresponding to the creation target direction into a predetermined fifth reference number (in this embodiment, five) of clusters by clustering the template material data in accordance with the distance matrix created in step S302 by a k-means method (step S303). After execution of the processing of step S303, the CPU 40 acquires the template material data of a centroid of each cluster as the template candidate corresponding to the creation target direction among the template material data corresponding to the creation target direction classified into the fifth reference number of clusters in step S303 (step S304).

In the description of this embodiment, the template material data is clustered by the k-means method in the processing of step S303, but this is merely an example, and any method can be used for clustering the template material data. For example, in the processing of step S303, the template material data may be clustered by the k-medoids method in accordance with the distance matrix created in step S302. In this case, in the processing of step S304, the template material data of the medoid of each cluster among the template material data clustered in step S303 may be acquired as the template candidate corresponding to the creation target direction.

After execution of the processing of step S304, the CPU 40 acquires the detection accuracy of the takeoff timing when each of the template candidates acquired in step S304 is used for detecting the takeoff timing in accordance with the test data corresponding to the creation target direction stored in the ROM 41 (step S305). Specifically, in the processing of step S305, the CPU 40 associates the test data corresponding to the creation target direction with the template candidate by the dynamic time warping method. All the template candidates are template material data, and include data indicating timing at which the subject performs the takeoff action. The CPU 40 detects the timing in the test data corresponding to the creation target direction corresponding to the timing indicated by the template candidate at which the subject performs the takeoff action as the takeoff timing. As described above, the test data includes the data indicating the timing at which the subject performs the takeoff action. The CPU 40 acquires a time difference between the takeoff timing detected by the above-described method in accordance with the test data and the template candidate and the timing indicated by the test data at which the subject performs the takeoff action as an index indicating detection accuracy of the takeoff timing when the template candidate is used for detecting the takeoff timing. The smaller the time difference is, the higher the detection accuracy of the takeoff timing is.

After execution of the processing of step S305, the CPU 40 adds data indicating the timing at which the subject performs the takeoff action to the template candidate having the highest detection accuracy of the takeoff timing acquired in step S305 among the template candidates acquired in step S304, and thus creates a template corresponding to the creation target direction (step S306). The template candidate having the highest detection accuracy of the takeoff timing is a template candidate having the smallest time difference between the takeoff timing detected with use of the template candidate and the timing indicated by the test data at which the takeoff action is performed. The timing at which the subject performs the takeoff action is a timing after the starting end of the template candidate as the template material data created by the above method by the fourth reference time, and before the terminal end of the template candidate by the fifth reference time. In the processing of step S306, the y-axis template corresponding to the y-axis direction is created when the creation target direction is the y-axis direction, and the z-axis template corresponding to the z-axis direction is created when the creation target direction is the z-axis direction. The template corresponding to the creation target direction created by the processing of step S306 indicates a typical temporal transition of the acceleration of the subject in the creation target direction when the subject performs the takeoff action.

After execution of the processing of step S306, the CPU 40 determines whether both the y-axis direction and the z-axis direction have already been set as the creation target directions (step S307). Upon determination that one of the y-axis direction or the z-axis direction has not yet been set as the creation target direction (step S307; No), the CPU 40 sets a direction that has not yet been set as the creation target direction of the y-axis direction or the z-axis direction as the creation target direction (step S308), and the processing returns to step S302. On the other hand, upon determination that both the y-axis direction and the z-axis direction have already been set as the creation target directions (step S307; Yes), the CPU 40 ends the template creation processing. As described above, until determining that both the y-axis direction and the z-axis direction have already been set as the creation target directions (step S307; Yes), the CPU 40 repeatedly executes the processing of steps S302 to S307, and thus creates both the y-axis template corresponding to the y-axis direction and the z-axis template corresponding to the z-axis direction.

The detection device 2 communicates with the information processor 4 with use of the communicator 25 to take in the y-axis template and the z-axis template created by the information processor 4 performing the above-described template creation processing from the information processor 4, and stores the y-axis template and the z-axis template in the template storage 205 provided in the storage area of the ROM 23.

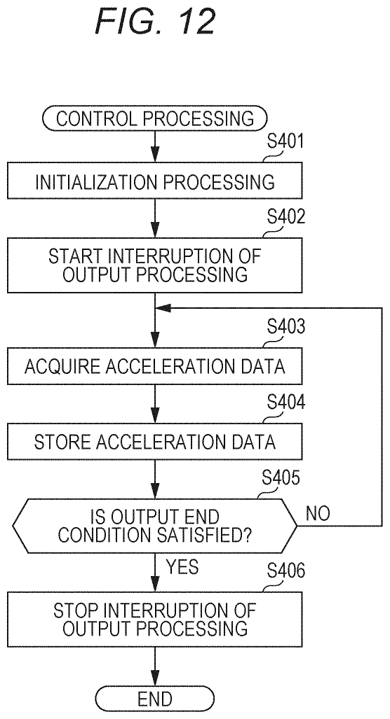

Hereinafter, processing executed by the detection device 2 having the above-described physical and functional configurations will be described with reference to flowcharts in FIGS. 12 to 15. Note that FIGS. 12 to 15 illustrate only processing related to a characteristic part of the present invention in the processing executable by the detection device 2. The detection device 2 can execute any processing not illustrated in FIGS. 12 to 15. For example, the detection device 2 may execute display control processing that controls display of an image by the display device.

First, control processing executed by the detection device 2 will be described with reference to the flowchart in FIG. 12. The detection device 2 acquires the takeoff type estimator created by the information processor 4 from the information processor 4 by communicating with the information processor 4 with use of the communicator 25, and stores the takeoff type estimator in advance in the estimator storage 203 provided in the storage area of the ROM 23. The detection device 2 acquires the y-axis template and the z-axis template created by the information processor 4 from the information processor 4 by communicating with the information processor 4 with use of the communicator 25, and stores the y-axis template and the z-axis template in advance in the template storage 205 provided in the storage area of the ROM 23. The detection device 2 acquires the y-axis prefilter and the z-axis prefilter created by the information processor 4 from the information processor 4 by communicating with the information processor 4 with use of the communicator 25, and stores the y-axis prefilter and the z-axis prefilter in advance in the prefilter storage 206 provided in the storage area of the ROM 23. The detection device 2 is fixed to the center of the waist of the user by the belt 21. In this state, when the user turns on the power source of the detection device 2 by operating the detection device power key included in the operation unit 26 of the detection device 2, the CPU 22 of the detection device 2 starts the control processing illustrated in the flowchart in FIG. 12.

Upon start of the control processing, the CPU 22 first executes initialization processing (step S401). In the initialization processing of step S401, the CPU 22 clears storage contents of the RAM 24. Further, in the initialization processing of step S401, the CPU 22 performs processing of setting each parameter used in output processing to be described later to a predetermined value, and thereafter, determines whether each parameter is actually set to the predetermined value. Upon determination that each parameter is set to a predetermined value, the processing proceeds to step S402. Upon determination that the parameter is set to a value different from the predetermined value, the CPU 22 resets each parameter to the predetermined value, and the processing proceeds to step S402.

After executing the processing of step S401, the CPU 22 starts interruption of the output processing to be described later (step S402). Thereafter, until the interruption of the output processing is stopped in the processing of step S406 described later, the CPU 22 interrupts the processing with the output processing upon each detection that a predetermined interruption cycle (in this embodiment, 1.5 s) has elapsed on the basis of the clock signal input from the clock unit 27, and repeatedly executes the output processing. Details of the output processing will be described later.

After execution of the processing of step S402, the acceleration data acquirer 200 acquires acceleration data indicating the temporal transition of the acceleration of the user in accordance with the acceleration signal input from the acceleration sensor 28a (step S403). In the processing of step S403, the acceleration data acquirer 200 uses Kalman filter to estimate the vertical direction and the advancing direction of the user in accordance with the detection result of the acceleration indicated by the acceleration signal input from the acceleration sensor 28a and the detection result of the angular velocity indicated by the angular velocity signal input from the angular velocity sensor 28b, and thus converts an acceleration value in a sensor coordinate system detected by the acceleration sensor 28a into an acceleration value in the world coordinate system defined by the above xyz coordinate axes. The acceleration data acquirer 200 acquires acceleration data by sampling the converted acceleration value at a predetermined sampling frequency (in this embodiment, 200 Hz). The acceleration data acquired in step S403 includes the x-axis acceleration data, y-axis acceleration data, and z-axis acceleration data. After execution of the processing of step S403, the acceleration data acquirer 200 stores the acceleration data acquired in step S403 in the acceleration data storage 201 (step S404).

After execution of the processing of step S404, the CPU 22 determines whether a predetermined output end condition is satisfied (step S405). In this embodiment, the output end condition is satisfied when the user turns off the power source of the detection device 2 by operating the detection device power key. Note that this is merely an example, and the output end condition can be arbitrarily set. For example, the output end condition may be set to be satisfied when an error occurs during the execution of the control processing. Upon determination that the output end condition is not satisfied in the process of step S405 (step S405; No), the processing returns to step S403.

On the other hand, upon determination that the output end condition is satisfied (step S405; Yes), the CPU 22 stops the interruption of the output processing (step S406), and ends the control processing. With such a configuration, until determination that the output end condition is satisfied (step S405; Yes), the processing of steps S403 and S404 is repeatedly executed, and every time an acceleration signal is input from the acceleration sensor 28a, acceleration data corresponding to the acceleration signal is acquired and accumulated in the acceleration data storage 201.

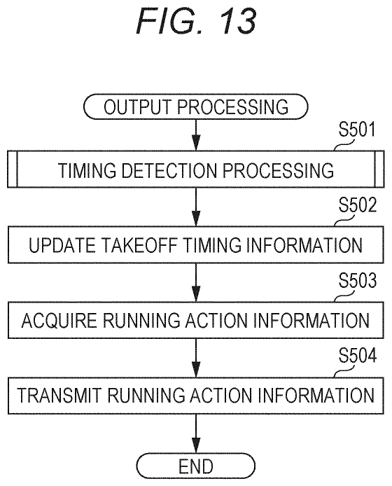

Hereinafter, details of the output processing will be described with reference to the flowchart in FIG. 13.

Upon start of the output processing, the CPU 22 first detects the takeoff timing by executing timing detection processing (step S501). Details of the timing detection processing will be described later. After execution of the processing of step S501, the timing detector 204 adds data indicating the takeoff timing detected in step S501 to the takeoff timing information stored in the takeoff timing information storage 207, and thus updates the takeoff timing information (step S502). After execution of the processing of step S502, the running action information acquirer 208 acquires the running action information indicating the average takeoff interval described above on the basis of the takeoff timing information updated in step S502 (step S503). After execution of the processing of step S503, the running action information output unit 209 controls the communicator 25 to transmit the running action information acquired in step S503 to the terminal device 3 (step S504), and ends the output processing.

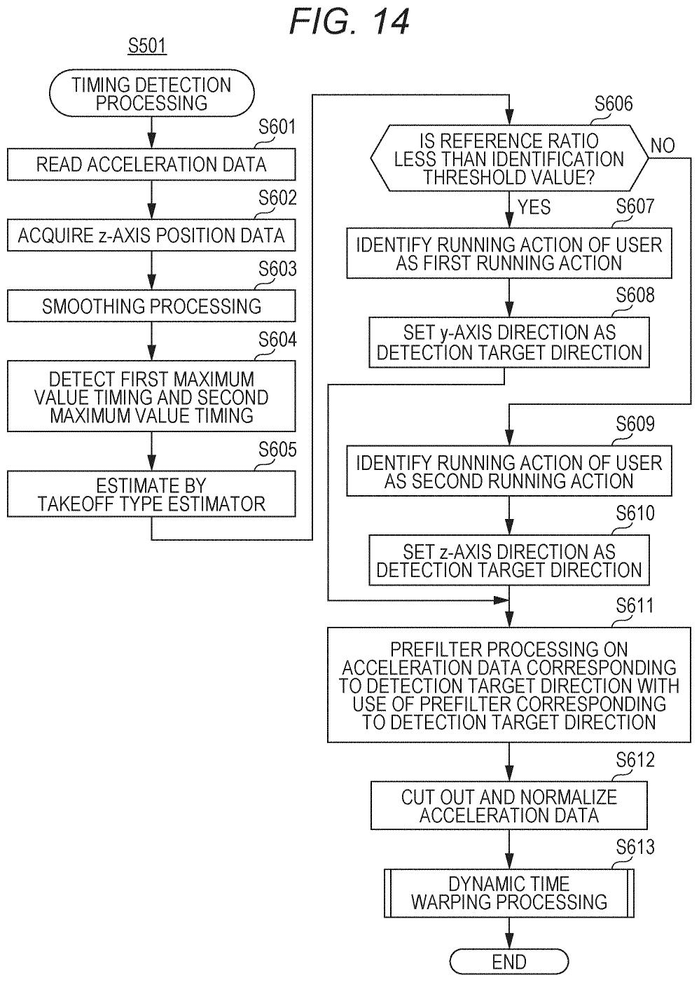

Hereinafter, details of the timing detection processing executed in step S501 of the output processing will be described with reference to the flowchart in FIG. 14.

Upon start of the timing detection processing, first, the running action identifier 202 reads, from the acceleration data storage 201, acceleration data corresponding to a processing period that is a time section from time earlier than the current time by the interruption cycle until the current time (step S601). The acceleration data read in step S601 is acceleration data corresponding to a period from the start of the previous timing detection processing to the start of the current timing detection processing. The acceleration data read in step S601 includes the x-axis acceleration data, y-axis acceleration data, and z-axis acceleration data.

The running action identifier 202 acquires the z-axis position data indicating the temporal transition of the position of the waist of the user in the z-axis direction by integrating the z-axis acceleration data included in the acceleration data read in step S601 twice (step S602). In the processing of step S602, the z-axis position data indicating the temporal transition of the position of the waist of the user in the z-axis direction in the above-described processing period is acquired. After execution of the processing of step S602, the running action identifier 202 executes the smoothing processing with use of the moving average filter on the z-axis position data acquired in step S602 (step S603). After execution of the processing of step S603, the running action identifier 202 detects the first maximum value timing at which the position of the waist of the user becomes a maximum value and the second maximum value timing at which the position of the waist of the user becomes a maximum value next to the first maximum value timing in the temporal transition of the position of the waist of the user in the z-axis direction in accordance with the z-axis position data subjected to the smoothing processing in step S603 (step S604). In the processing of step S604, in the temporal transition of the position of the waist of the user in the z-axis direction in the processing period, the timing at which the position of the waist of the user first reaches the maximum value is detected as the first maximum value timing.

After execution of the processing of step S604, the running action identifier 202 inputs, as a feature amount, the acceleration of the user in the x-axis direction, the y-axis direction, and the z-axis direction at the timing that is indicated by the acceleration data read in step S601 and that is before the second maximum value timing detected in step S604 by the first reference time (in this embodiment, 500 ms) to the takeoff type estimator stored in the estimator storage 203, and thus causes the takeoff type estimator to estimate whether the reference ratio corresponding to the takeoff timing is less than the identification threshold value (in this embodiment, 0.95) (step S605).

After execution of the processing of step S605, the running action identifier 202 determines whether the reference ratio is estimated to be less than the identification threshold value in step S605 (step S606). Upon determination that the reference ratio is estimated to be less than the identification threshold value (step S606; Yes), the running action identifier 202 identifies the running action of the user as the first running action (step S607). After execution of the processing of step S607, the timing detector 204 sets the y-axis direction as the detection target direction (step S608). On the other hand, upon determination that the reference ratio is estimated to be the identification threshold value or more in the processing of step S606 (step S606; No), the running action identifier 202 identifies the running action of the user as the second running action (step S609). After execution of the processing of step S609, the timing detector 204 sets the z-axis direction as the detection target direction (step S610).

After execution of the processing in step S608 or step S610, the timing detector 204 executes the prefilter processing on the acceleration data corresponding to the detection target direction set in step S608 or step S610 with use of the prefilter corresponding to the detection target direction, of the y-axis prefilter or the z-axis prefilter stored in the prefilter storage 206 (step S611). In a case where the y-axis direction is set as the detection target direction in step S608, the prefilter processing is performed on the y-axis acceleration data corresponding to the y-axis direction with use of the y-axis prefilter corresponding to the y-axis direction in the processing of step S611. In a case where the z-axis direction is set as the detection target direction in step S610, the prefilter processing is performed on the z-axis acceleration data corresponding to the z-axis direction with use of the z-axis prefilter corresponding to the z-axis direction in the processing of step S611. After execution of the processing of step S611, the timing detector 204 cuts out acceleration data belonging to a range from the first maximum value timing to the second maximum value timing detected in step S604 from the acceleration data corresponding to the detection target direction subjected to the prefilter processing in step S611, and normalizes the cut acceleration data (step S612). In the processing of step S612, the acceleration indicated by the cut acceleration data is normalized to a range from −10 to 10, and a time range from the first maximum value timing to the second maximum value timing indicated by the acceleration data is normalized to a range from 0 to 100.

After execution of the processing of step S612, the timing detector 204 executes dynamic time warping processing to detect the takeoff timing by associating the acceleration data corresponding to the detection target direction cut out and normalized in step S612 with the template corresponding to the detection target direction by the dynamic time warping method (step S613), and ends the timing detection processing. In the processing of step S607, when the running action identifier 202 identifies the running action of the user as the first running action (step S607), the y-axis direction is set as the detection target direction (step S608), and in the processing of step S613, the takeoff timing is detected in accordance with the y-axis acceleration data corresponding to the y-axis direction as the detection target direction and the y-axis template corresponding to the y-axis direction. That is, when the running action of the user is identified as the first running action (step S607), the takeoff timing is detected by the first detection method. On the other hand, when the running action identifier 202 identifies the running action of the user as the second running action in the processing of step S609 (step S609), the z-axis direction is set as the detection target direction (step S610), and the takeoff timing is detected in accordance with the z-axis acceleration data corresponding to the z-axis direction as the detection target direction and the z-axis template corresponding to the z-axis direction in the processing of step S613. That is, when the running action of the user is identified as the second running action (step S609), the takeoff timing is detected by the second detection method.

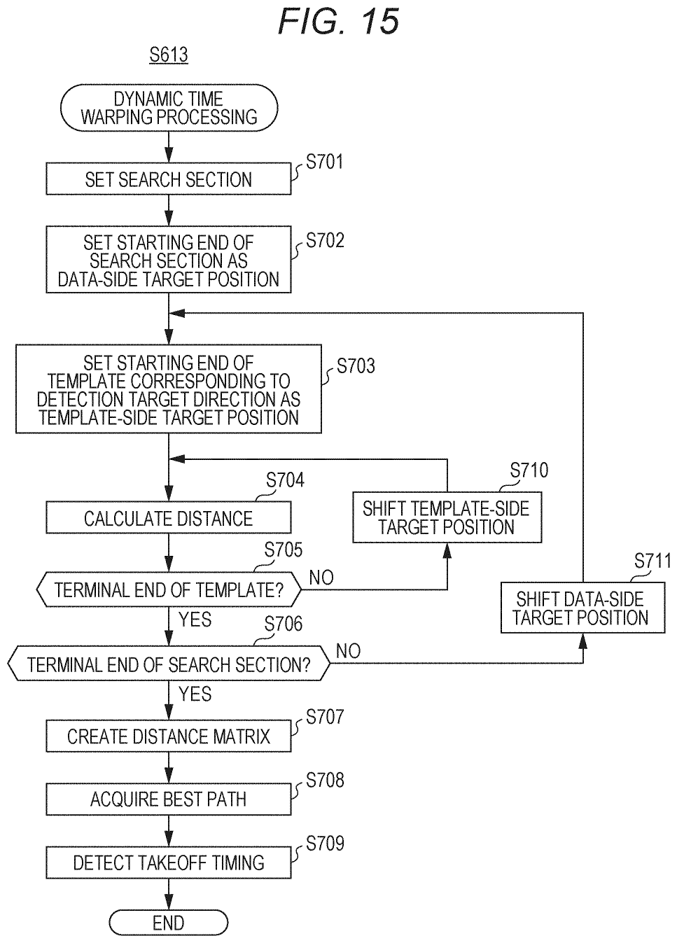

Hereinafter, details of the dynamic time warping processing executed in step S613 of the timing detection processing will be described with reference to the flowchart in FIG. 15.

Upon start of the dynamic time warping processing, the timing detector 204 first sets a search section in the acceleration data and cut out and normalized in step S612 of the flowchart in FIG. 14 in accordance with the first maximum value timing and the second maximum value timing detected in step S604 (step S701). In the processing of step S701, a section from the timing before the second maximum value timing by the second reference time to the second maximum value timing is set as the search section. The second reference time is time obtained by multiplying the time from the first maximum value timing to the second maximum value timing by the above-described reference multiplier (in this embodiment, 0.5).

After execution of the processing of step S701, the timing detector 204 sets a sampling point at a starting end of the search section set in step S701 as a data-side target position (step S702). After execution of the processing of step S702, the timing detector 204 sets a sampling point at a starting end of the template corresponding to the detection target direction set in step S608 or step S610 of the flowchart in FIG. 14 as a template-side target position (step S703).

After execution of the processing of step S703, the timing detector 204 calculates a distance between data corresponding to the data-side target position in the data included in the acceleration data corresponding to the detection target direction cut out and normalized in step S612 and data corresponding to the template-side target position in the data included in the template corresponding to the detection target direction, and stores data indicating the calculated distance in a predetermined area of the RAM 24 (step S704). In the processing of step S704, an absolute value of a difference between the data is calculated as the distance between the data. In the processing of step S704, data indicating the calculated distance is stored in association with data indicating the currently set data-side target position and the currently set template-side target position.

After execution of the processing of step S704, the timing detector 204 determines whether the currently set template-side target position is a sampling point at a terminal end of the template corresponding to the detection target direction (step S705). Upon determination that the currently set template-side target position is the sampling point at the terminal end of the template corresponding to the detection target direction (step S705; Yes), the processing proceeds to step S706. On the other hand, upon determination that the currently set template-side target position is not the sampling point at the terminal end of the template corresponding to the detection target direction (step S705; No), the timing detector 204 shifts the template-side target position to the next sampling point of the currently set template-side target position in the template corresponding to the detection target direction (step S710), and the processing returns to step S704. Until determining that the currently set template-side target position is the sampling point at the terminal end of the template corresponding to the detection target direction (step S705; Yes), the timing detector 204 repeatedly executes the processing of steps S704 and S705 while shifting the template-side target position (step S710), and thus calculates the distance between the data of the currently set data-side target position among the acceleration data included in the search section set in step S701 and each data of all the sampling points of the template corresponding to the detection target direction, and accumulates the distance in the predetermined area of the RAM 24.

In the processing of step S706, the timing detector 204 determines whether the currently set data-side target position is the sampling point at the terminal end of the search section set in step S701 (step S706). Upon determination that the currently set data-side target position is the sampling point at the terminal end of the search section (step S706; Yes), the processing proceeds to step S707. On the other hand, upon determination that the currently set data-side target position is not the sampling point at the terminal end of the search section (step S706; No), the timing detector 204 shifts the data-side target position to the next sampling point of the currently set data-side target position in the acceleration data included in the search section (step S711), and the processing returns to step S703. Until determining that the currently set data-side target position is the sampling point at the terminal end of the search section (step S706; Yes), the timing detector 204 repeatedly executes the processing of steps S703 to S706 while shifting the data-side target position (step S711), and thus calculates the distance between each data of all sampling points of the acceleration data included in the search section and each data of all sampling points of the template corresponding to the detection target direction, and accumulates the distance in the predetermined area of the RAM 24.

In the processing of step S707, the timing detector 204 creates a distance matrix indicating the distance between the data of each sampling point of the acceleration data included in the search section and the data of each sampling point of the template corresponding to the detection target direction in accordance with the data that is accumulated in the predetermined area of the RAM 24 by repeatedly executing the processing of step S704 and indicates the distance between the data of each sampling point of the acceleration data included in the search section and the data of each sampling point of the template corresponding to the detection target direction (step S707).

After execution of the processing of step S707, the timing detector 204 obtains the best path indicating the correspondence relationship between the acceleration data included in the search section and the template corresponding to the detection target direction in accordance with the distance matrix created in step S707, and thus associates the acceleration data with the template (step S708). In the processing of step S708, a path having the smallest sum of elements of the distance matrix on the path is obtained as the best path among the paths on the distance matrix created in step S707, in which an element of the distance matrix indicating the distance between data that is included in the acceleration data and corresponds to the starting end of the search section and data that is included in the template corresponding to the detection target direction and corresponds to the starting end of the template is set as a starting end, and an element of the distance matrix indicating the distance between data that is included in the acceleration data and corresponds to the terminal end of the search section and data that is included in the template and corresponds to the terminal end of the template is set as a terminal end.

After execution of the process of step S708, the timing detector 204 detects, as the takeoff timing, the timing in the acceleration data corresponding to the timing indicated by the template at which the subject performs the takeoff action corresponding to the detection target direction (step S709), and ends the dynamic time warping processing.

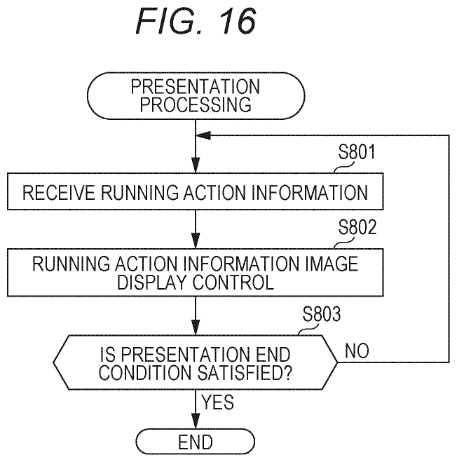

Next, the presentation processing executed by the terminal device 3 having the above-described physical and functional configurations will be described with reference to the flowchart in FIG. 16. Note that FIG. 16 illustrates only processing related to a characteristic part of the present invention in the processing executable by the terminal device 3. The terminal device 3 can execute any processing not illustrated in FIG. 16. For example, the terminal device 3 may execute a voice output control processing that controls voice output by the speaker described above.

Hereinafter, the description will be made by exemplifying a case where the detection device 2 is turned on in a state where the detection device 2 is attached to the waist of the user, and the detection device 2 transmits the running action information to the terminal device 3 by executing the above-described output processing for each interruption cycle. In this state, when the user inputs an instruction to start the presentation of the running action information to the terminal device 3 by operating the touch screen included in the operation unit 34 of the terminal device 3, the CPU 30 of the terminal device 3 starts the presentation processing illustrated in the flowchart in FIG. 16.

Upon start of the presentation processing, the running action information receiver 300 controls the communicator 33 to receive the running action information transmitted from the communicator 25 of the detection device 2 in accordance with the control by the running action information output unit 209 (step S801). After execution of the processing of step S801, the running action information presenter 301 performs running action information image display control of causing the display unit 35 to display a running action information image corresponding to the running action information received in step S801 (step S802). When the display unit 35 has already displayed the running action information image, the running action information image display control is executed in step S802, and thus the running action information image currently displayed by the display unit 35 is updated to a new running action information image corresponding to the running action information received in step S801.

After execution of the processing of step S802, the CPU 30 determines whether a predetermined presentation end condition is satisfied (step S803). In this embodiment, the presentation end condition is satisfied when the user inputs an instruction to end the presentation of the running action information to the terminal device 3 by operating the touch screen included in the operation unit 34 of the terminal device 3. Note that this is merely an example, and the presentation end condition can be arbitrarily set. For example, the presentation end condition may be set to be satisfied when the user turns off the power of the terminal device 3 by operating the terminal device power key included in the operation unit 34. Alternatively, the presentation ending condition may be set to be satisfied when predetermined time (for example, 10 minutes) has elapsed without the communicator 33 receiving the running action information from the detection device 2. Upon determination that the presentation ending condition is not satisfied in the processing of step S803 (step S803; No), the processing returns to step S801. On the other hand, upon determination that the presentation end condition is satisfied (step S803; Yes), the CPU 30 ends the presentation processing. With such a configuration, until determining that the presentation ending condition is satisfied (step S803; Yes), the CPU 30 repeatedly executes the processing of steps S801 to S803, and updates the running action information image displayed by the display unit 35 every time the running action information is received from the detection device 2.

As described above, when the running action of the user is the first running action, the timing detector 204 detects the takeoff timing with use of a first detection method, and when the running action of the user is the second running action, the timing detector detects the takeoff timing with use of a second detection method. The detection target direction and the template used for detection when the timing detector 204 detects the takeoff timing by the first detection method are different from the detection target direction and the template used for detection when the timing detector 204 detects the takeoff timing by the second detection method. This configuration can improve the detection accuracy of the takeoff timing.

Further, the timing detector 204 associates the acceleration data corresponding to the detection target direction with the template corresponding to the detection target direction by the dynamic time warping method, and detects, as the takeoff timing, the timing in the acceleration data corresponding to the timing indicated by the template at which the subject performs the takeoff action. This configuration can improve the detection accuracy of the takeoff timing by detecting the takeoff timing robustly against variations in the running speed of the user.

Further, the timing detector 204 detects the takeoff timing by associating the data included in the search section among the acceleration data corresponding to the detection target direction with the template corresponding to the detection target direction by the dynamic time warping method. This configuration can reduce the calculation load and improve the detection accuracy of the takeoff timing.

The timing detector 204 detects the takeoff timing in accordance with the y-axis template when detecting the takeoff timing by the first detection method, and detects the takeoff timing in accordance with the z-axis template when detecting the takeoff timing by the second detection method. This configuration can improve the detection accuracy of the takeoff timing by detecting the takeoff timing with use of an appropriate template in accordance with the type of the running action of the user.

Further, the timing detector 204 detects the takeoff timing in accordance with the y-axis acceleration data when detecting the takeoff timing by the first detection method, and detects the takeoff timing in accordance with the z-axis acceleration data when detecting the takeoff timing by the second detection method. This configuration can improve the detection accuracy of the takeoff timing by detecting the takeoff timing with use of the acceleration data corresponding to the appropriate detection target direction in accordance with the type of the running action of the user.

The timing detector 204 detects the takeoff timing by the first detection method when the running action identifier 202 identifies the running action of the user as the first running action, and detects the takeoff timing by the second detection method when the running action identifier 202 identifies the running action of the user as the second running action. This configuration can improve the detection accuracy of the takeoff timing by detecting the takeoff timing with use of an appropriate detection method in accordance with the type of the running action of the user.

Further, the running action identifier 202 identifies whether the running action of the user is the first running action or the second running action in accordance with the magnitude relationship between the reference ratio and the identification threshold value. This configuration can improve the identification accuracy of the type of the running action of the user and improve the detection accuracy of the takeoff timing by detecting the takeoff timing by an appropriate detection method in accordance with the type of the running action of the user.

Further, the running action information acquirer 208 acquires the running action information in accordance with the takeoff timing detected by the timing detector 204, and the running action information output unit 209 outputs the running action information acquired by the running action information acquirer 208. This configuration can improve the accuracy of the running action information and convenience for the user.

Although the embodiment of the present invention has been described above, the present invention is not limited to the above embodiment, and various modifications can be made without departing from the gist of the present invention.

For example, in the above embodiment, it has been described that the first running action is the running action in a case where the user is running at the first reference speed as the average running speed of adults, and the second running action is the running action in a case where the user is running at the second reference speed that is slower than the first reference speed, but this is merely an example. The first running action and the second running action may be any running actions different from each other. For example, the first running action may be a running action in a case where the user performs a short distance running such as 50 meters running or 100 meters running, and the second running action may be a running action in a case where the user performs a long distance running such as a half marathon or a full marathon.

In the above embodiment, it has been described that an example of the first moving action is the first running action, and an example of the second moving action is the second running action. However, this is merely an example, and the first moving action may be the running action, and the second moving action may be the walking action.

In the above embodiment, it has been described that both the detection target direction and the template used for detecting the takeoff timing are different between when the timing detector 204 detects the takeoff timing by the first detection method and when the timing detector 204 detects the takeoff timing by the second detection method. However, this is merely an example. One of the detection target direction or the template used for detecting the takeoff timing may be different between when the timing detector 204 detects the takeoff timing by the first detection method and when the timing detector 204 detects the takeoff timing by the second detection method.

For example, the templates used for detecting the takeoff timing may be different between when the timing detector 204 detects the takeoff timing by the first detection method and when the timing detector 204 detects the takeoff timing by the second detection method, but the detection target directions may be the same.

Hereinafter, a modification will be described in which the timing detector 204 performs detection by setting the y-axis direction as the detection target direction whether the timing detector 204 detects the takeoff timing by the first detection method or the second detection method. In this modification, the template storage 205 stores in advance, as templates, a first running action template indicating a temporal transition of the acceleration of the subject in the y-axis direction when the subject who is moving by performing the first running action performs the takeoff action, and a second running action template indicating a temporal transition of the acceleration of the subject in the y-axis direction when the subject who is moving by performing the second running action performs the takeoff action. The first running action template and the second running action template each include data indicating the timing at which the subject performs the takeoff action. The first running action template is an example of the first reference data, and the second running action template is an example of the second reference data.

The first running action template and the second running action template are created by the information processor 4, then taken in by the detection device 2, and stored in advance in the template storage 205. The information processor 4 creates the first running action template by executing processing similar to the template creation processing of the above embodiment by using, as the template material data, the data indicating the temporal transition of the acceleration of the subject in the y-axis direction when the subject who is moving by performing the first running action performs the takeoff action. The first running action template created by such a method shows a typical temporal transition of the acceleration of the subject in the y-axis direction when the subject who is moving by performing the first running action performs the takeoff action. Further, the information processor 4 creates the second running action template by executing processing similar to the template creation processing of the above embodiment by using, as the template material data, the data indicating the temporal transition of the acceleration of the subject in the y-axis direction when the subject who is moving by performing the second running action performs the takeoff action. The second running action template created by such a method shows a typical temporal transition of the acceleration of the subject in the y-axis direction when the subject who is moving by performing the second running action performs the takeoff action.

The timing detector 204 performs detection with use of the first running action template when detecting the takeoff timing by the first detection method, and performs detection with use of the second running action template when detecting the takeoff timing by the second detection method. That is, when detecting the takeoff timing by the first detection method, the timing detector 204 associates the y-axis acceleration data corresponding to the y-axis direction as the detection target direction with the first running action template by the dynamic time warping method, and detects, as the takeoff timing, the timing in the y-axis acceleration data corresponding to the timing indicated by the first running action template at which the subject performs the takeoff action. On the other hand, when detecting the takeoff timing by the second detection method, the timing detector 204 associates the y-axis acceleration data corresponding to the y-axis direction as the detection target direction with the second running action template by the dynamic time warping method, and detects, as the takeoff timing, the timing in the y-axis acceleration data corresponding to the timing indicated by the second running action template at which the subject performs the takeoff action. This configuration can improve the detection accuracy of the takeoff timing by detecting the takeoff timing with use of an appropriate template in accordance with the type of the running action of the user.

In this modification, it has been described that the y-axis direction is set as the detection target direction in both the first detection method and the second detection method, but this is merely an example. In both the first detection method and the second detection method, the z-axis direction may be set as the detection target direction. Such a modification can be realized by replacing the “y-axis direction” in the modification with the “z-axis direction”.

The modification has been described in which the templates used for detecting the takeoff timing is different between when the timing detector 204 detects the takeoff timing by the first detection method and when the timing detector 204 detects the takeoff timing by the second detection method, but the detection target directions is the same. However, this is merely an example. The detection target direction may be different between when the timing detector 204 detects the takeoff timing by the first detection method and when the timing detector 204 detects the takeoff timing by the second detection method, and the templates used for detecting the takeoff timing may be the same.

Hereinafter, a modification will be described in which the timing detector 204 performs detection with use of an identical common template whether the timing detector detects the takeoff timing by the first detection method or the second detection method. In this modification, the common template indicating a temporal transition of the acceleration of the subject in the y-axis direction and the z-axis direction when the subject who is moving by performing the running action performs the takeoff action is stored in the template storage 205 in advance as a template. The common template includes data indicating the timing at which the subject performs the takeoff action.

The common template is created by the information processor 4, then taken in by the detection device 2, and stored in advance in the template storage 205. The information processor 4 creates the common template by executing processing similar to the template creation processing of the above embodiment with use of, as template material data, data indicating a temporal transition of the acceleration of the subject in the y-axis direction when the subject who is moving by performing the running action performs the takeoff action and data indicating a temporal transition of the acceleration of the subject in the z-axis direction when the subject who is moving by performing the running action performs the takeoff action. The common template created by such a method indicates a typical temporal transition of the acceleration of the subject in the y-axis direction and the z-axis direction when the subject who is moving by performing the running action performs the takeoff action.

The timing detector 204 performs detection by setting the y-axis direction as the detection target direction when detecting the takeoff timing by the first detection method, and performs detection by setting the z-axis direction as the detection target direction when detecting the takeoff timing by the second detection method. That is, when detecting the takeoff timing by the first detection method, the timing detector 204 associates the y-axis acceleration data corresponding to the y-axis direction as the detection target direction with the common template by the dynamic time warping method, and detects, as the takeoff timing, the timing in the y-axis acceleration data corresponding to the timing indicated by the common template at which the subject performs the takeoff action. On the other hand, when detecting the takeoff timing by the second detection method, the timing detector 204 associates the z-axis acceleration data corresponding to the z-axis direction as the detection target direction with the common template by the dynamic time warping method, and detects, as the takeoff timing, the timing in the z-axis acceleration data corresponding to the timing indicated by the common template at which the subject performs the takeoff action. This configuration can improve the detection accuracy of the takeoff timing by setting an appropriate direction as the detection target direction in accordance with the type of the running action of the user and detecting the takeoff timing. Further, this configuration eliminates the need for storing a different template for each detection method and can reduce a storage load. Furthermore, this configuration eliminates the need for creating a different template for each detection method and can reduce a manufacturing cost of the detection device 2.

In the above embodiment, it has been described that the timing detector 204 detects the takeoff timing by either the first detection method or the second detection method in accordance with the identification result of the running action of the user by the running action identifier 202, but this is merely an example. The detection device 2 can detect the takeoff timing by a detection method corresponding to the type of the running action of the user without identifying whether the running action of the user is the first running action or the second running action.

Specifically, hereinafter, a modification will be described in which the timing detector 204 detects two candidate timings that are candidates for the takeoff timing by both the first detection method and the second detection method, and determines one of the two candidate timings as the takeoff timing.

In this modification, the timing detector 204 detects a first candidate timing as one candidate for the takeoff timing by the first detection method. Specifically, the timing detector 204 creates a distance matrix indicating a distance between data of each sampling point of the y-axis acceleration data corresponding to the y-axis direction as the detection target direction in the first detection method and data of each sampling point of the y-axis template corresponding to the y-axis direction, and obtains the best path indicating the correspondence relationship between the y-axis acceleration data and the y-axis template in accordance with the distance matrix by the dynamic time warping method. The timing detector 204 thus associates the y-axis acceleration data with the y-axis template and detects, as the first candidate timing, a timing in the y-axis acceleration data corresponding to the timing indicated by the y-axis template at which the subject performs the takeoff action. Furthermore, the timing detector 204 acquires a sum of the elements of the distance matrix on the obtained best path as an index indicating a similarity between the y-axis acceleration data and the y-axis template.

The timing detector 204 detects a second candidate timing as another candidate for the takeoff timing by the second detection method. Specifically, the timing detector 204 creates a distance matrix indicating a distance between data of each sampling point of the z-axis acceleration data corresponding to the z-axis direction as the detection target direction in the second detection method and data of each sampling point of the z-axis template corresponding to the z-axis direction, and obtains the best path indicating the correspondence relationship between the z-axis acceleration data and the z-axis template in accordance with the distance matrix by the dynamic time warping method. The timing detector 204 thus associates the z-axis acceleration data with the z-axis template and detects, as the second candidate timing, a timing in the z-axis acceleration data corresponding to the timing indicated by the z-axis template at which the subject performs the takeoff action. Furthermore, the timing detector 204 acquires a sum of the elements of the distance matrix on the obtained best path as an index indicating a similarity between the z-axis acceleration data and the z-axis template.

The timing detector 204 determines, as the takeoff timing, one of the first candidate timing detected by the first detection method or the second candidate timing detected by the second detection method in accordance with the similarity between the acceleration data corresponding to the detection target direction and the template corresponding to the detection direction. In this modification, the sum of the elements of the distance matrix on the best path indicating the correspondence relationship between the acceleration data corresponding to the detection direction obtained by the dynamic time warping method and the template corresponding to the detection direction is used as an index indicating the similarity between the acceleration data and the template. The smaller the sum of the elements on the best path indicating the correspondence relationship between the acceleration data and the template, the higher the similarity between the acceleration data and the template.

Specifically, when the sum of the elements of the distance matrix on the best path indicating the correspondence relationship between the y-axis acceleration data and the y-axis template is smaller than the sum of the elements of the distance matrix on the best path indicating the correspondence relationship between the z-axis acceleration data and the z-axis template, that is, when the similarity between the y-axis acceleration data and the y-axis template is higher than the similarity between the z-axis acceleration data and the z-axis template, the timing detector 204 determines the first candidate timing detected by the first detection method as the takeoff timing. On the other hand, when the sum of the elements of the distance matrix on the best path indicating the correspondence relationship between the y-axis acceleration data and the y-axis template is greater than or equal to the sum of the elements of the distance matrix on the best path indicating the correspondence relationship between the z-axis acceleration data and the z-axis template, that is, when the similarity between the y-axis acceleration data and the y-axis template is lower than or equal to the similarity between the z-axis acceleration data and the z-axis template, the timing detector 204 determines the second candidate timing detected by the second detection method as the takeoff timing.

When the running action of the user is the first running action, the similarity between the y-axis acceleration data and the y-axis template is higher than the similarity between the z-axis acceleration data and the z-axis template. Thus, when the running action of the user is the first running action, the timing detector 204 determines, as the takeoff timing, the first candidate timing detected by the first detection method. In other words, when the running action of the user is the first running action, the timing detector 204 detects the takeoff timing by the first detection method. When the running action of the user is the second running action, the similarity between the z-axis acceleration data and the z-axis template is higher than the similarity between the y-axis acceleration data and the y-axis template. Thus, when the running action of the user is the second running action, the timing detector 204 determines, as the takeoff timing, the second candidate timing detected by the second detection method. In other words, when the running action of the user is the second running action, the timing detector 204 detects the takeoff timing by the second detection method.

As described above, the configuration of this modification makes it possible to detect the takeoff timing by an appropriate detection method according to the type of the running action of the user without identifying whether the running action of the user is the first running action or the second running action, and to improve the detection accuracy of the takeoff timing. This configuration can reduce the possibility of deteriorating the detection accuracy of the takeoff timing by detecting the takeoff timing by the detection method corresponding to the type of the running action erroneously identified because of erroneous identification of the type of the running action of the user.

It should be noted that not only a dedicated measurement device having a configuration for realizing each function of the present invention in advance can be provided as the measurement device of the present invention, but also application of a program enables an existing measurement device to function as the measurement device of the present invention. That is, by applying the program for realizing each function of the measurement device of the present invention so as to be executable by a processor such as a CPU that controls the existing measurement device, the existing measurement device can function as the measurement device of the present invention.

Note that a method of applying such a program is arbitrary. The program can be stored and applied in a computer-readable recording medium such as a flexible disk, a compact disc (CD)-ROM, a digital versatile disc (DVD)-ROM, or a memory card. Furthermore, the program may be superimposed on a carrier wave and applied via a communication medium such as the Internet. For example, the program may be posted and distributed on a bulletin board system (BBS) on a communication network. Then, the above processing may be executed by starting the program and executing the program in the same manner as other application programs under the control of an operating system (OS).

Although the preferred embodiment of the present invention has been described above, the present invention is not limited to the specific embodiment, and the present invention includes the invention described in the claims and the equivalent scope thereof. Hereinafter, the invention described in the original claims of the present application will be described.

What is claimed is:

1. A measurement device comprising: at least one processor configured to execute a program stored in at least one memory, wherein
the at least one processor acquires acceleration data indicating a temporal transition of acceleration of a subject when the subject is moving by performing a moving action,
the at least one processor detects a target timing that is a timing when the subject performs a target action in accordance with a temporal transition of the acceleration of the subject in a detection target direction, the temporal transition being indicated by the acceleration data, and in accordance with reference data indicating a temporal transition of the acceleration of the subject when the subject performs the target action in the moving action,
the at least one processor detects the target timing using a first detection method when the moving action is a first moving action,
the at least one processor detects the target timing using a second detection method different from the first detection method when the moving action is a second moving action different from the first moving action, and
at least one of the detection target direction or the reference data is different between when the target timing is detected using the first detection method and when the target timing is detected using the second detection method.

2. The measurement device according to claim 1, wherein
the target action is a takeoff action of taking a foot off from a ground or a landing action of landing a foot separated from the ground on the ground.

3. The measurement device according to claim 1, wherein
the reference data includes data indicating the timing at which the subject performs the target action,
the processor associates the acceleration data with the reference data using a dynamic time warping method, and
the processor detects, as the target timing, a timing in the acceleration data corresponding to the timing indicated by the reference data at which the subject performs the target action.

4. The measurement device according to claim 3, wherein
the processor associates data included in a search section among the acceleration data with the reference data using the dynamic time warping method.

5. The measurement device according to claim 1, wherein
the memory stores, as the reference data, first reference data and second reference data different from the first reference data, and
the processor detects the target timing in accordance with the first reference data when detecting the target timing using the first detection method, and detects the target timing in accordance with the second reference data when detecting the target timing using the second detection method.

6. The measurement device according to claim 1, wherein
the acceleration data includes first acceleration data indicating a temporal transition of acceleration of the subject in a direction parallel to an advancing direction of the subject and second acceleration data indicating a temporal transition of acceleration of the subject in a direction parallel to a vertical direction, and
the processor detects the target timing in accordance with the first acceleration data when detecting the target timing using the first detection method, and detects the target timing in accordance with the second acceleration data when detecting the target timing using the second detection method.

7. The measurement device according to claim 1, wherein
the processor identifies whether the moving action is the first moving action or the second moving action, and
the processor detects the target timing using the first detection method when the moving action is identified as the first moving action, and detects the target timing using the second detection method when the moving action is identified as the second moving action.

8. The measurement device according to claim 7, wherein
the processor identifies whether the moving action is the first moving action or the second moving action in accordance with a magnitude relationship between a reference ratio and a predetermined identification threshold value,
the reference ratio is a ratio of time from a first timing until the target timing with respect to time from the first timing until a second timing,
the first timing is a timing at which a position of a part of a body of a subject becomes a maximum value in a temporal transition of the position of the part of the body in a direction parallel to a vertical direction, and
the second timing is a timing at which the position of the part of the body becomes the maximum value next to the first timing in the temporal transition.

9. The measurement device according to claim 1, wherein
the processor
detects a first candidate timing using the first detection method,
detects a second candidate timing using the second detection method,
determines the first candidate timing as the target timing when the moving action is the first moving action, and
determines the second candidate timing as the target timing when the moving action is the second moving action.

10. The measurement device according to claim 1, wherein
the processor acquires moving action information indicating a feature of the moving action in accordance with the target timing that has been detected, and outputs the moving action information that has been acquired.

11. A measurement method, comprising the steps of:
acquiring acceleration data indicating a temporal transition of acceleration of a subject when the subject is moving by performing a moving action;
detecting a target timing that is a timing when the subject performs a target action in accordance with the temporal transition of the acceleration of the subject in a detection target direction, the temporal transition being indicated by the acceleration data, and in accordance with reference data indicating the temporal transition of the acceleration of the subject when the subject performs the target action in the moving action;
detecting the target timing using a first detection method when the moving action is a first moving action; and
detecting the target timing using a second detection method different from the first detection method when the moving action is a second moving action different from the first moving action, wherein
at least one of the detection target direction or the reference data is different between when the target timing is detected using the first detection method and when the target timing is detected using the second detection method.

12. A non-transitory recording medium storing a program for causing a computer to execute:
an acceleration data acquisition function of acquiring acceleration data indicating a temporal transition of acceleration of a subject when the subject is moving by performing a moving action; and
a timing detection function of detecting a target timing that is a timing when the subject performs the target action in accordance with a temporal transition of the acceleration of the subject in the detection target direction, the temporal transition being indicated by the acceleration data, and in accordance with reference data indicating a temporal transition of the acceleration of the subject when the subject performs the target action in the moving action, wherein
the timing detection function includes
detecting the target timing using a first detection method when the moving action is a first moving action, and
detecting the target timing using a second detection method different from the first detection method when the moving action is a second moving action different from the first moving action, and
at least one of the detection target direction or the reference data is different between when the target timing is detected using the first detection method in the timing detection function and when the target timing is detected using the second detection method in the timing detection function.
